(19) United States
(12) Patent Application Publication
(10) Pub. No.: US 2024/0011092 A1
(43) Pub. Date: Jan. 11, 2024

(54) USE OF TORQUE TENO VIRUS (TTV) AS A MARKER TO DETERMINE THE PROLIFERATIVE CAPACITY OF T LYMPHOCYTES

(71) Applicants: BIOMERIEUX, Marcy L'etoile (FR); HOSPICES CIVILS DE LYON, Lyon (FR); UNIVERSITE CLAUDE BERNARD LYON 1, Villeurbanne (FR)

(72) Inventors: Karen BRENGEL-PESCE, Les Abrets En Dauphine (FR); Sophie ASSANT-TROUILLET, Reyrieux (FR); William MOUTON, Serezin-Du-Rhone (FR)

(73) Assignees: BIOMERIEUX, Marcy L'etoile (FR); HOSPICES CIVILS DE LYON, Lyon (FR); UNIVERSITÉ CLAUDE BERNARD LYON 1, Villeurbanne (FR)

(21) Appl. No.: 18/035,239

(22) PCT Filed: Nov. 4, 2021
(86) PCT No.: PCT/FR2021/051951

§ 371 Date: May 3, 2023

(30) Foreign Application Priority Data
Nov. 4, 2020 (FR) FR2011328

(51) Int. Cl. C12Q 1/6881; C12Q 1/70

(52) U.S. Cl. CPC C12Q 1/6881; C12Q 1/701; C12Q 2600/158

(57) ABSTRACT

The subject matter is a method for determining the proliferation capacity of T lymphocytes in a subject, the method including the following steps: a) measuring the load of torque teno virus from a biological sample of the subject; and b) determining the proliferative capacity of the T lymphocytes based on the viral load measured in a).

### CROSS-REFERENCE TO RELATED APPLICATIONS

This application is a U.S. national phase entry of International Patent Application No. PCT/FR2021/051951, filed on Nov. 4, 2021, which claims priority to French Patent Application Serial No. FR2011328, filed on Nov. 4, 2020, both of which are incorporated by reference herein.

### BACKGROUND AND SUMMARY

The immune system defends the body against attack such as pathogenic infections, cellular transformation and physical or chemical damage. An immunocompetent individual is thus capable of triggering a protective immune response against antigen stimulation.

However, when the immune system is weakened or totally absent, an immunodeficiency is manifested. This assumes various forms and may affect the innate or the adaptive immune system, or both, depending on the source of the deficit.

In the vast majority of cases, immunodeficiency is acquired throughout life. It may result from a pathology, whether or not infectious (for example HIV infection), or it may be induced by therapy, such as radiotherapy or chemotherapy. The immunodeficiency state is particularly dangerous, as the patient then displays increased susceptibility to secondary infections by pathogens such as bacteria, viruses, parasites or fungi. For example, the use of immunosuppressive treatments for grafts, in particular hematopoietic stem cell transplantation (HSCT), may lead to recurrent or potentially fatal microbial infections in patients.

It is therefore important to be able to determine the functionality of a subject's immune system. This makes it possible in particular to adapt an appropriate therapeutic response when an immunodeficiency is identified.

Various techniques are available for measuring immunocompetence, but none is completely satisfactory. These techniques measure among other things the immune response to cellular mediation and comprise in particular analysis of the populations of lymphocytes, in particular the count of CD4⁺ T cells or measurement of the ratio of CD4⁺/CD⁺ T cells, measurement of the capacity for lymphocyte proliferation, measurement of the cytotoxic activity of T cells, measurement of the antibody response, labeling of tetramers, detection of the cytokines produced, ELISpot, etc.

Some of these techniques, for example such as counting of T cells, give a result that does not necessarily reflect the activity of these cells and therefore the activity of the immune system. An absolute number of T cells says nothing about the capacity of these cells to multiply. For example, it cannot be said that a small number of T cells after HSCT signifies that these T cells are incapable of multiplying and of defending the patient against a microbial infection. Moreover, cellular proliferation tests are difficult to implement routinely and may be difficult to standardize. There is therefore still a need for a simple method that is easy to use for determining immunocompetence.

The Torque Teno Virus (TTV) is a virus of the family Anelloviridae initially identified in 1997 in a Japanese patient with post-transfusion hepatitis (1-7). TTV is a virus with single-stranded circular DNA of small size (about 3.8 kb), comprising a coding region having great genetic diversity and a well conserved noncoding region (UTR). The use of primers for amplifying sequences in this noncoding region has demonstrated a high global prevalence of TTV (around 90%). TTV causes chronic infections without clearly associated clinical manifestation. It is called a nonpathogenic or orphan virus. Numerous studies have thus dealt with the involvement of TTV in human pathology, in particular in certain hepatic pathologies, without a clear role being identified for this virus.

It has been observed, however, that the TTV load is higher in subjects with an immunodeficiency. For example, high levels of TTV are found in patients who have received immunosuppressant treatments in the context of organ transplants (Rezahosseini et al., Transplant Rev (Orlando). 33(3): 137-144, 2019) or of HSCT (Albert et al., Med Microbiol Immunol. 208(2): 253-258, 2019). Moreover, there would seem to be a relation between the TTV load and deficiencies of the immune system associated with chronic infections or with cancer (Zhong et al., Ann NY Acad Sci. 945: 84-92, 2001; Fogli et al., Clin Dev Immunol. 2012: 829584, 2012; Béland et al., J infect Dis. 209(2): 247-254, 2014; Görzer et al., J Heart Lung Transplant. 33(3): 320-323, 2014), whereas TTV is found in association with immunosuppressive viral infections such as HIV or HCV infections.

These observations led to the suggestion that the TTV load could be a marker of immunocompetence (8-11). However, in these studies, immunocompetence was only evaluated from the number of immune cells or from the occurrence of undesirable clinical events (11-14). Now, it has already been suggested that the quantity of cells is not necessarily associated with the quality of the cells, i.e. the activity of T cells is not reflected in their number (12).

There is therefore still a need for a simple, reliable method that makes it possible to determine whether T cells can be activated in a subject.

### BRIEF DESCRIPTION OF THE DRAWINGS

FIG. 1 shows TTVs structure and genomic organization;

FIG. 2 shows TTV viral load from plasma samples from recipients of allo-HSCT and from healthy volunteers;

FIGS. 3A-D show a correlation between the TTV viral load and T cell count as well as the proliferative capacity of the CD3+ T cells;

FIG. 4 shows chronological descriptive monitoring of the patients with extreme values of TTV viral load; and

FIG. 5 shows correlation between the TTV viral load and the times since HSCT.

### DETAILED DESCRIPTION

The present invention relates to a method for determining whether T cells are functional in a subject. More precisely, the inventors have shown that the proliferative capacity of the T cells is inversely correlated with the load of torque teno virus (TTV), in particular in a patient who has received a transplant, more particularly HSCT. The TTV viral load is inversely correlated with proliferation of T cells: thus, the higher the TTV load, the lower the proliferative capacity of the T cells. The viral load is not correlated specifically with another parameter, whether this is the number of T cells or a clinical criterion, emphasizing the relevance of the correlation identified. The TTV load is a specific marker of the activity of T cells, in particular in patients who have received a transplant and in particular HSCT. The development of the functionality of the immune system may therefore be traced in a subject by simply measuring their TTV load. It is thus possible to evaluate the functional state of the immune system quickly without having to implement the onerous technical steps usually employed for evaluating this parameter.

### Methods of Determining the Proliferative Capacity of T Cells

The present description relates to a method of determining the proliferative capacity of T cells in a subject, said method comprising measurement of the TTV load in the patient.

As mentioned above, the proliferative capacity of T cells is not necessarily correlated with the number of said T cells. Consequently, the known methods for counting T cells are not in any way informative about the proliferative capacity of T cells and therefore about the functionality of the immune system. Therefore the inventors have the merit of having identified a new parameter, easily measurable routinely, which is advantageously correlated with the proliferative capacity of T cells, thus making it possible to evaluate the functionality of the immune system.

The “T cells” or “T cells” are essential cells of the immune system tasked with amplifying or reducing the immune response. Preferably, T cells are characterized by expression of a membrane marker called CD3 and a specific receptor, TCR (for “T cell receptors”), which is directly involved in antigen recognition. Advantageously, the T cells may express other surface markers, in particular CD4 and CD8, which correspond to specific functional categories of T cells. In the context of HSCT, the T cells involved may in particular be the few residual T cells of the recipient, or the donor's T cells present in the transplant. They may also be naive T cells resulting from the differentiation of the donor's stem cells and progenitors in the recipient's thymus.

Clean Substitute Specification 5

The expression “activation of the T cells” or “activation of T cells” refers here to the process by which naive T cells become able to participate in the immune response. Activation of T cells leads in particular to their proliferation. Activation of T cells is thus advantageously evaluated by measuring the proliferation of T cells. Measurement of the proliferation of T cells is usually carried out by techniques that are familiar to a person skilled in the art, but are onerous to implement. In particular, it is well known that T cells are able to proliferate in the presence of a mitogen, for example such as concanavalin A (Con A), the mitogen of pokeweed (PWM for “pokeweed mitogen”) and phytohaemagglutinin (PHA), independently of the specificity of their TCR. The methods of the prior art evaluate the proliferative capacity of the T cells by measuring the synthesis of the DNA of the T cells after stimulation thereof by a mitogen. However, these methods are onerous to implement and may thus be difficult to use routinely. In comparison, the method described here is particularly simple and robust.

Thus, T cell proliferation test may comprise the following steps of:
isolating the peripheral blood mononuclear cells (PBMC) from whole blood by centrifugation,
incubating said isolated PBMC, optionally in a supplemented medium, for example in a cell culture plate,
stimulation, preferably double, with a mitogen,
incubation, and
assay by flow cytometry, for example from the culture pellets, in order to determine the proliferation of the T cells.

A detailed protocol is given in particular in the embodiment examples.

According to a first aspect, a method is described here for determining the proliferative capacity of T cells in a subject, said method comprising:
a) measurement of the TTV load from a sample from said subject; and
b) determining the proliferative capacity of the subject's T cells in view of the viral load measured in a).

According to a preferred embodiment, a high TTV load indicates that T cells have a low proliferative capacity. Conversely, a low TTV load indicates that the cells have a high proliferative capacity.

Of course, in order to determine whether the TTV load in the biological sample is high or low, and draw a conclusion about the proliferative capacity of T cells, said TTV load may advantageously be compared with a reference TTV load or control load as defined later in the present description. As an example, a reference TTV load may be the TTV viral load measured in one and the same individual.

The present method is suitable more particularly for evaluating the proliferative capacities of T cells in a subject who may have an immunodeficiency state.

The term “immunodeficiency” (or “immunodepression” or “immunosuppression”), as used here, refers to the reduction or suppression of the function of the immune system. Here, an “immunodeficiency state” therefore denotes a state in which a subject's immune system is reduced or absent. Preferably, the humoral and/or cellular immune response to infectious pathogens is defective in subjects with an immunodeficiency state. More preferably, the immunodeficiency state is manifested at least by a decrease in the cellular response.

Immunodeficiency may be primary or secondary. The primary immunodeficiencies include the innate deficits of the immune system with an increased susceptibility to infections. In contrast, secondary immunodeficiency (or acquired immunodeficiency) corresponds to a loss of the immune function that appears during life, such as, for example but without limitation, following exposure to pathogens, a disease (for example lymphoma or leukemia), therapy for treating a disease (for example, radiotherapy or chemotherapy), immunosuppression, or aging. The pathogens that may lead to immunodeficiency comprise, among others, the human immunodeficiency virus (HIV) 1 (HIV-1), HIV-2, Treponema pallidum, Plasmodium falciparum, Plasmodium malariae, Plasmodium ovale, Plasmodium vivax, Plasmodium knowlesi, hepatitis B virus (HBV), hepatitis C virus (HCV), prions, West Nile virus, parvovirus, Trypanosoma cruzi, coronaviruses such as SARS-CoV-1 and SARS-CoV-2, and/or the cowpox virus. Immunodeficiency may also be induced deliberately with drugs, for example in preparation for a transplant, for example such as an organ transplant (for example of kidney, liver, heart, lung, pancreas, intestine, etc.) or HSCT, to prevent graft rejection.

Preferably, the immunodeficiency according to the present description is a secondary (or acquired) immunodeficiency. Thus, the immunodeficiency described here may be of any origin such as, for example, but without limitation, an immunosuppressant treatment, immunosuppressant side effects of medication or of therapy comprising radiotherapy, hereditary immunosuppressant genetic traits or diseases, acquired immunosuppressive diseases such as AIDS, cancers, such as leukemia or lymphoma. In particular, the immunodeficiency is associated with a transplant, in particular HSCT.

In a particular embodiment, the subject who is likely to have an immunodeficiency state is a subject who has received a transplant. According to a more particular embodiment, this transplant is HSCT.

More particularly, the description relates to a method for determining the proliferative capacity of T cells in a subject, the subject having received HSCT, the method comprising the steps of:
a) measuring the TTV load from a sample from the subject; and
b) determining the proliferative capacity of the subject's T cells in view of the viral load measured in a).

According to a preferred embodiment, a high TTV load indicates that the cells have a low proliferative capacity. Conversely, a low TTV load indicates that the cells have a high proliferative capacity.

Here, “stem cell” is intended to denote undifferentiated but specialized cells having two main properties: the capacity for self-renewal and to remain in place for very long periods, and the capacity to generate all the types of differentiated cells of a specific tissue, which defines their pluripotency. Here, the “hematopoietic stem cells” or “HSC” denote more particularly the stem cells that may lead to the different cells of the blood (in particular red blood cells, platelets, granulocytes, T or B cells, and monocytes). The HSC may advantageously be obtained from umbilical cord blood. Alternatively, they may be obtained from the peripheral blood. It is also possible to obtain them from bone marrow.

“Hematopoietic stem cell transplantation” or “HSCT”, as it is to be understood here, is a therapeutic procedure in the field of hematology in which HSCs, generally derived from the bone marrow, peripheral blood or umbilical cord blood, are transplanted from a donor to a recipient.

HSCT is a potentially therapeutic approach for a variety of diseases. These are in particular hemopathies, in particular malignant hemopathies, such as acute leukemias, myelodysplasias and lymphomas, and nonmalignant hemopathies with a severe prognosis, including constitutional medullar aplasias and hemoglobinopathies, solid tumors, immune deficits and enzymatic deficits of the hematopoietic tissue, such as Gaucher disease, for example. Preferably, the pathology is a hemopathy, more preferably a malignant hemopathy.

The HSCT may be autologous (the patient's own stem cells are used, i.e. the donor and the recipient are one and the same person) or allogenic (“allo-HSCT” hereinafter: the stem cells are derived from a donor who is not the recipient). Preferably, HSCT in the method described here is an allogenic transplant.

According to this particular embodiment, the description relates to a method for determining the proliferative capacity of T cells in a subject, the subject having undergone an allo-HSCT, the method comprising steps of:
a) measuring the TTV load from a sample from the subject; and
b) determining the proliferative capacity of the subject's T cells in view of the viral load measured in a).

According to a preferred embodiment, a high TTV load indicates that the cells have a low proliferative capacity. Conversely, a low TTV load indicates that the cells have a high proliferative capacity.

In the case of an allograft, treatments for preparation (or conditioning treatment) are administered before transplantation to destroy or to reduce the activity of the recipient's immune system. This conditioning aims to prevent graft rejection and reduce the tumoral load.

The conditioning may be myeloablative. A “myeloablative” conditioning, as it is understood here, is a conditioning that destroys the cells of the recipient's bone marrow. Advantageously, the myeloablative conditioning also destroys the recipient's immune system, thus facilitating graft acceptance. The myeloablative conditioning may in particular comprise one or more steps of chemotherapy and/or radiotherapy. For example, two of the conditionings commonly used are the combination busulfan-cyclophosphamide and cyclophosphamide-total body irradiation (TBI). Preferably, myeloablative conditioning is applied for a patient under 55 years, preferably under 50 years.

Alternatively, the conditioning is a nonmyeloablative conditioning, attenuated, also called “reduced-intensity”. An “attenuated conditioning” is a conditioning that does not destroy the recipient's bone marrow completely, but leads to inhibition of their immune system, thus facilitating graft acceptance. This attenuated conditioning preferably comprises administration of an immunosuppressant. For example, a protocol of attenuated conditioning may comprise a combination of fludarabine, cyclophosphamide or another alkylating agent, and of a TBI. Another example of a protocol of attenuated conditioning may comprise the combination of fludarabine, antilymphocyte serum (ALS) and busulfan. Another example of a protocol of attenuated conditioning may comprise the combination of fludarabine, idarubicin and cytarabine. Finally, another example of a protocol of attenuated conditioning may comprise the combination of fludarabine with a complete mini-irradiation. In a preferred embodiment, attenuated conditioning is applied for a patient under 75 years.

The term “donor”, as used here, refers to the subject whose HSCs are transferred to the recipient. Here, “recipient” or “patient” means the subject who receives the HSCs from the donor. In a particular embodiment, the recipient is affected by a pathology for which HSCT should provide a therapeutic benefit, whether complete or partial.

As used here, the term “subject” refers to a vertebrate, preferably a mammal and, most preferably of all, a human. A human may for example be a patient.

Preferably, in the methods as described hereunder, in all their embodiments, the subject is a patient.

The term “patient” denotes a human being who has contacted a health professional, such as a doctor, a medical structure or a healthcare establishment such as a hospital for example.

Here, “biological sample” means any sample that may be taken from a subject. In general, the biological sample must allow determination of the TTV load. The biological sample, as it is understood here, comprises among other things, but is not limited to, whole blood, serum, plasma, expectorations, nasopharyngeal samples, urine, feces, skin, cerebrospinal fluid, saliva, gastric secretions, sperm, seminal fluid, tears, spinal tissue or fluid, cerebral fluid, a sample of trigeminal ganglion, a sample of sacral lymph node, adipose tissue, lymphoid tissue, placental tissue, tissue of the upper reproductive system, tissue of the gastrointestinal system, genital tissue and tissue of the central nervous system. The test sample may be used directly from the biological source or following a pretreatment for modifying the character of the sample. For example, this pretreatment may include preparation of plasma from blood, dilution of viscous fluids and so on. Methods of pretreatment may also involve filtration, precipitation, dilution, distillation, mixing, concentration, inactivation of disturbing components, addition of reagents, lysis, etc. Furthermore, it may be beneficial to modify a solid test sample to form a liquid medium or to release the analyte. Preferably, the biological sample is blood or a derivative thereof, such as plasma or serum. Thus, the biological sample is preferably blood, plasma or serum derived from the subject being tested.

“Torque teno virus or Torque Teno virus or TTV” means a virus of the family Anelloviridae. A TTV, as it is understood here, is a nonenveloped virus, with a genome of circular single-stranded DNA of negative polarity. “TTV genome” refers here to the genomes of all the Anelloviridae, including the alphatorqueviruses (TTV), the betatorqueviruses (TTMV), the gammatorqueviruses (TTMDV). As an example, reference is made here to the genome of the prototype strain of the Torque Teno virus, TTV-1a. More specifically, an example of a TTV genome is a sequence such as for example that represented by SEQ ID No 1 and whose Genbank accession number is AB017610.

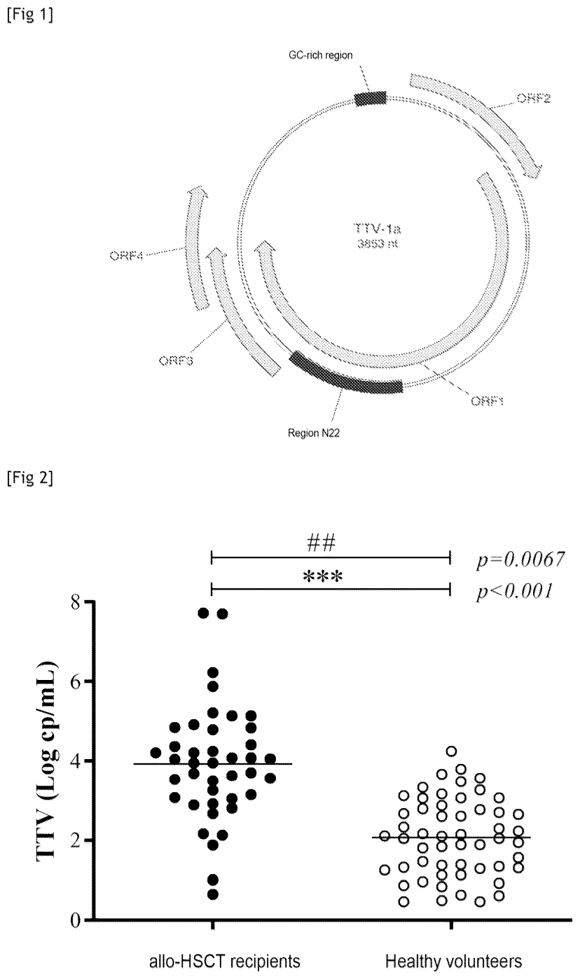

Preferably, the TTV genome has a size of about 3.8 kb. The structure and the genomic organization of the TTVs is well known (cf. for example Biagini, Curr Top Microbiol Immunol. 331: 21-33, 2009) and is exemplified in [FIG. 1]. The TTV genome may thus be divided into an untranslated region (UTR) of about 1 to 1.2 kb and a potential coding region of about 2.6 to 2.8 kb. The coding region contains in particular two large open reading frames: ORF1 and ORF2, coding for two proteins with 770 and 202 residues respectively. In the TTV genome represented by SEQ ID No 1, the open reading frames ORF1 and ORF2 are between nucleotides 589-2901 and 107-715, respectively. The TTV genome may have other open reading frames. For example, the TTV genome may comprise two additional reading frames, ORF3 and ORF4 [FIG. 1 ].

In contrast, the untranslated region UTR is well conserved. It comprises in particular a sequence rich in GC that may form a secondary structure. The amplification of sequences selected in the untranslated region UTR-5′ made it possible to demonstrate that the prevalence of the virus is very high throughout the whole world population (Hu et al., J Clin Microbiol. 43(8): 3747-3754, 2005). This region comprises in particular a sequence of 128 bp that can be amplified with the TTV R-GENE® diagnostic kit (bioMérieux, France).

The “viral load”, as it is understood here, is the number of viral particles present in a biological sample. The viral load reflects the severity of a viral infection. The viral load may be determined by measuring the amount of one of the components of the virus (genomic DNA, mRNA, protein etc.) in this biological sample. Preferably, the viral load thus refers to the proportion of nucleic acid sequences belonging to said virus in a biological sample. More preferably, the viral load represents the number of copies of the genome of said virus in a biological sample.

In the present case, the viral load represents the TTV load. The “TTV load” corresponds here more particularly to the viral load of the TTV, i.e. the number of viral particles of TTV present in a biological sample. The TTV load in a subject means the viral load of all TTV harbored by said subject. The TTV load may be determined by measuring the amount of a component of TTV, such as a nucleic acid or a protein, in this biological sample. Preferably, the TTV load corresponds to the quantity of nucleic acid sequences of TTV present in a biological sample. Thus, determination of the TTV load in a subject according to the invention comprises estimation of the number of sequences of all the TTVs in a biological sample from said subject. In particular, there is no selection, according to the invention, of specific strains of TTV to be measured in said biological sample. Preferably, determination of the TTV load comprises determination of the quantity of active and/or inactive viral copies. It consists of determining the quantity of integrated or latent circulating viral copies.

The levels of TTV—and therefore the TTV load - may be determined by measuring the levels of TTV DNA, of TTV RNA or of TTV proteins. The method according to the invention may thus comprise another preliminary step, between taking of the sample from the patient and step a) as defined above, corresponding to transformation of the biological sample into a sample of double-stranded DNA, or into a sample of mRNA (or of corresponding cDNA), or into a sample of proteins, which is then ready to be used for in vitro detection of TTV in step a). The preparation or extraction of double-stranded viral DNA, of mRNA (as well as the reverse transcription of the latter into cDNA) or of proteins starting from a cellular sample are merely routine procedures familiar to a person skilled in the art. The double-stranded DNA may correspond either to the whole of the TTV genome, or only to a part thereof. Once a double-stranded DNA, an mRNA (or a corresponding cDNA) or a ready-to-use sample of proteins is available, detection of the TTVs can be carried out, depending on the type of sample or of transformation, either by the genomic DNA (i.e. based on the presence of at least one sequence consisting of at least a part of the TTV genome), or by the mRNA (i.e. based on the content of TTV mRNA in the sample), or at the protein level (i.e. based on the content of TTV proteins in the sample).

Preferably, the levels of TTV are determined by measuring the levels of TTV nucleic acid, more preferably of TTV DNA.

The methods of detection of a nucleic acid in a biological sample comprise, among other things, amplification, including PCR amplification, sequencing, hybridization with a labeled probe and all the other methods known by a person skilled in the art.

According to a first embodiment, the TTV load is determined by amplification of the TTV sequences.

A preferred approach consists of amplifying sequences that are known to be specific to the TTV genome. Here, “sequence specific to the TTV genome” means a sequence that is present in the majority of the known TTVs, but that is absent from the majority of the other viruses, in particular the majority of other anelloviruses. Preferably, a sequence specific to TTV is present in at least 90%, at least 95%, at least 96%, at least 97%, at least 98% or at least 99% of the genomes of known TTVs. More preferably, it is present in 100% of the genomes of known TTVs. Alternatively, a sequence specific to the TTVs is present in less than 10%, less than 5%, less than 4%, less than 3%, less than 2%, less than 1% of the genomes of known anelloviruses other than the TTVs. Preferably, a sequence specific to the TTVs is absent from all the genomes of known anelloviruses other than the TTVs. Such a sequence is for example a sequence comprised in the untranslated region UTR. More particularly, such a sequence corresponds to the sequence of 128 bp of the untranslated region 5′-UTR that is amplified using the TTV R-GENE® diagnostic kit (bioMérieux, France).

Thus, according to this embodiment, the method described here comprises the use of primers and probes for amplification of sequences known to be specific to the TTV genome. As is usual in this technical field, these primers are preferably oligonucleotides. For example, these primers may comprise less than 30 nucleotides, less than 25 nucleotides, less than 20 nucleotides, less than 15 nucleotides or less than 12 nucleotides. Alternatively, these primers comprise at least 12, 15, 20, 25 or 30 nucleotides. Preferably, the primers used comprise between 12 and 20 nucleotides, between 12 and 25 nucleotides, between 15 and 20 nucleotides or else between 15 and 25 nucleotides. A person skilled in the art will know how to determine the length and the sequence of the amplification primer to be used once the sequence specific to the TTVs has been selected. It will be possible for example to use the same primers as those that are supplied in the TTV R-GENE® diagnostic kit (bioMérieux, France).

The amplification techniques in particular comprise isothermal methods and techniques based on PCR (Polymerase Chain Reaction). The methods of isothermal amplification include a large number of methods. The methods most used for detecting pathogens are LAMP (Loop-Mediated Amplification) and RPA (Recombinase Polymerase Amplification). The methods of isothermal amplification also comprise methods such as for example NASBA (nucleic acid sequence-based amplification), HDA (helicase-dependent amplification), RCA (rolling circle amplification) and SDA (strand displacement amplification), EXPAR (exponential amplification reaction), ICANs (isothermal and chimeric primer-initiated amplification of nucleic acids), SMART (signal-mediated amplification of RNA technology), etc. (see for example Asiello and Baeumner, Lab Chip 11(8): 1420-1430, 2011). Preferably, the PCR technique used measures the initial quantities of DNA, cDNA or RNA quantitatively. Examples of techniques based on PCR that may be used in the methods described here comprise, without limitation, techniques such as real-time PCR (Q-PCR), reverse transcription PCR (RT-PCR), multiple reverse transcription PCR, real-time reverse transcription PCR (QRT-PCR) and digital PCR. These techniques are technologies that are well known and readily available for a person skilled in the art. It is not necessary to detail them here.

Preferably, the TTV load is determined by real-time quantitative PCR. Numerous methods for detection and quantification of the TTVs have been described in the prior art (see for example Maggi et al., J Virol. 77(4): 2418-2425, 2003). Reference will be made in particular to the method described by Kulifaj et al. (J Clin Virol. 105:118-127, 2018). This method is particularly advantageous owing to its simplicity and robustness. It is based on amplification of a sequence comprised in the noncoding region UTR. This sequence is present in all the known TTVs, thus endowing the method with very great specificity. Furthermore, it is particularly versatile and may be carried out with any type of PCR platform. It is particularly advantageous to use the TTV R-GENE® diagnostic kit (bioMérieux, France) for carrying out this method.

Alternatively, determination of the viral load is carried out by digital PCR. Digital PCR involves several PCR analyses on extremely diluted nucleic acids so that most of the positive amplifications reflect the signal of a single matrix molecule. Digital

PCR thus allows counting of individual model molecules. The proportion of positive amplifications among the total number of PCRs analyzed allows estimation of the concentration of matrix in the original or undiluted sample. This technique was proposed for allowing detection of a variety of genetic phenomena (Vogelstein et al., Proc Natl Acad Sci USA 96: 9236-924, 1999). Digital PCR, just like real-time PCR, potentially allows discrimination of fine quantitative differences of target sequences between samples.

According to another embodiment, the levels of TTV DNA are measured by sequencing. As used here, the term “sequencing” is taken in its widest acceptation and refers to any technique known by a person skilled in the art for determining the sequence of a polynucleotide molecule (DNA or RNA), i.e. for determining the succession of nucleotides making up this molecule.

The TTV DNA may thus be sequenced by any technique known in the art. Sequencing, as it is understood here, comprises among other things sequencing by the Sanger method, sequencing of the entire genome, sequencing by hybridization, pyrosequencing (in particular 454 sequencing, Solexa Genome Analyzer sequencing), sequencing with capillary electrophoresis, sequencing in cycles, single base extension sequencing, solid phase sequencing, high-throughput sequencing, massively parallel signature sequencing (MPSS), reversible dye terminator sequencing, sequencing with paired pairs, short term sequencing, sequencing with exonucleases, sequencing by ligation, single molecule sequencing, sequencing by synthesis, sequencing by electron microscopy, real time sequencing, reverse termination sequencing, sequencing by nanopores, reversible terminator sequencing, sequencing by semiconductor, SOLiD(R) sequencing, SMRT sequencing (Single Molecule Real-Time Analysis), MS-PET sequencing, mass spectrometry, and combinations thereof. A particular embodiment uses high-throughput DNA sequencing, for example using the platforms MiSeq, NextSeq 500, and the HiSeq series developed by Illumina (Reuter et al., Mol Cell, 58: 586-597, 2015; Bentley et al. Nature; 456: 53-59, 2008), the 454 Genome Sequencer and Roche platform (Margulies et al. Nature; 437: 376-380, 2005), the SOLiD platform of Applied Biosystems (McKernan et al., Genome Res; 19: 1527-1541, 2009), the Polanator platform (Shendure et al., Science, 309: 1728-1732) or the Helicos single molecule sequencing platform (Harris et al. Science; 320: 106-109, 2008). The high-throughput sequencing also includes methods such as SMRT real time sequencing (Roads et al., Genomics, Proteomics & Bioinformatics, 13(5): 278-289, 2015), Ion Torrent sequencing (WO 2010/008480; Rothberg et al., Nature, 475: 348-352, 2011) and sequencing using nanopores (Clarke J et al. Nat Nanotechnol: 4: 265-270, 2009).

The sequencing is carried out on the whole of the DNA contained in the biological sample or on parts of the DNA contained in the biological sample. It will immediately be clear to a person skilled in the art that said sample contains at least a mixture of TTV DNA and DNA of the host subject. Moreover, the TTV DNA will probably only represent a minor fraction of the total DNA present in the sample. Advantageously, the DNA is fragmented at random, generally by physical methods, prior to sequencing.

A first approach consists of sequencing specific sequences of the genome of a species of TTV. Another approach consists of using a method that allows quantitative genotyping of the nucleic acids obtained from the biological sample with great accuracy. In a particular embodiment, the accuracy is obtained by analyzing a large number (for example, millions or billions) of nucleic acid molecules without any amplification using protocols that are based on prior knowledge of the target sequences (i.e. in this case, the sequences of the TTVs).

In a preferred embodiment, the method of the invention comprises a step of quantification of the number of readings.

In a particular embodiment, a random subset of nucleic acid molecules of the biological sample is submitted to high-throughput sequencing. Preferably, the TTV sequences are identified in the global sequencing data by comparison with the publicly deposited TTV sequences. This comparison is based advantageously on the level of sequence identity with a known TTV sequence and makes it possible to detect even distant variants. Common software such as BLAST may be used for determining the level of identity between the sequences.

Thus, a sequence having at least 80%, at least 85%, at least 90%, at least 95%, at least 96%, at least 97%, at least 98%, at least 99% or 100% identity with a known TTV sequence is identified as a TTV sequence. According to this embodiment, determination of the TTV load therefore comprises numbering of the TTV sequences identified by sequencing in the biological sample from the subject.

In another embodiment, the TTV load is determined by measuring the amount of a viral protein in the biological sample. It is thus possible to use specific antibodies, in particular in well-known technologies such as immunoprecipitation, immunohistology, western blot, dot blot, ELISA or ELISPOT, electrochemiluminescence (ECLIA), protein chips, antibody chips, or tissue chips coupled to immunohistochemistry. Other techniques that may be used include the FRET or BRET techniques, methods of microscopy or of histochemistry, including in particular the methods of confocal microscopy and electron microscopy, methods based on the use of one or more excitation wavelengths and an adapted optical method, such as an electrochemical method (voltammetry and amperometry techniques), the atomic force microscope, and radiofrequency methods, such as multipolar, confocal, and nonconfocal resonance spectroscopy, detection of fluorescence, luminescence, chemiluminescence, absorbance, reflectance, transmittance, and birefractive power or refractive index (for example by surface plasmon resonance, by ellipsometry, by the resonant mirror method, etc.), flow cytometry, radioisotopic or magnetic resonance imaging, analysis by electrophoresis in polyacrylamide gel (SDS-PAGE); by mass spectrometry and by liquid chromatography coupled to mass spectrometry (LC-MS/MS). All these techniques are familiar to a person skilled in the art and it is not necessary to detail them here. Furthermore, specific antibodies of TTV proteins are already available.

In a preferred embodiment, the method described here comprises an additional step of normalization of the quantity of nucleic acid or of viral protein measured.

According to a preferred embodiment, it may be advantageous to normalize the levels of TTV, i.e. the quantity of TTV DNA, of TTV RNA or of TTV proteins present in the biological sample to a specific parameter of this sample. Normalization of the measured TTV load to a specific parameter makes it possible to reduce the error rate when comparing the viral loads of two different biological samples. An example of a parameter that may be useful for this normalization may be a physical parameter that is independent of the contents of the sample, for example such as the volume of the latter. It is also possible to normalize the quantity of TTV DNA, of TTV RNA or of TTV proteins to the total amount of DNA, of RNA or of proteins present in the sample. Alternatively, it is possible to use a particular DNA or RNA sequence or a particular protein as a normalization tool. For example, this sequence or this protein may be a human sequence or protein.

Alternatively, the quantity of TTV DNA or RNA or of TTV proteins in a given sample is compared against an internal control. For this, the quantity of TTV nucleic acid or protein measured in the biological sample may be referred to a defined quantity of a nucleic acid or of a suitable protein that may be identified and quantified, for example such as a host or exogenous nucleic acid or protein. Preferably, this identifiable and quantifiable nucleic acid or protein is treated (for example, amplified, sequenced, etc.) as the target nucleic acid or protein. It is thus possible ab initio to add to the sample a known quantity of this identifiable and quantifiable nucleic acid or protein, which will then be treated as the target nucleic acid or protein and will pass through all the steps of sample preparation prior to measurement of the quantity of this viral nucleic acid or protein. The preparation steps may comprise means for protecting the viral nucleic acid and destroying the host nucleic acids, for example using various nucleases. Alternatively, these steps may comprise means for protecting the viral proteins and destroying the host proteins, for example using various proteases. The internal control makes it possible to evaluate the quality and the extent of the treatment (for example an amplification or a sequencing) of the molecules under consideration (nucleic acids or proteins) in the sample. Preferably, said internal control is a nucleic acid molecule of known sequence, this nucleic acid molecule being present in the sample at a known concentration. More preferably, this nucleic acid molecule is the molecule of genomic single-stranded circular DNA of a virus of known sequence and concentration in the sample. This known virus may be for example a virus of the family Circoviridae. The ratio of the number of sequences of the sample to the control makes it possible to estimate the absolute number of TTV genomes of known sequence and concentration. Alternatively, this internal control is a protein of known sequence, which is present in the sample at a known concentration.

Once the TTV load has been determined by measuring the quantity of TTV nucleic acid or protein determined, the latter optionally being normalized, it may be advantageous to compare it against a reference TTV load.

“A reference TTV load” or “a reference viral load” means, in the sense of the present application, any TTV load used as a reference. This means that the reference TTV load corresponds to “a reference level of TTV nucleic acid (or protein)” or “a control level of TTV nucleic acid (or protein)”, i.e. to a concentration of a TTV nucleic acid (or protein) used as a reference. As it is understood here, “a reference concentration of TTV nucleic acid (or protein)” is a baseline level measured in a control sample comparable to that tested, and which is obtained from a subject or from a group of subjects having a specific immunocompetent status. It may be for example a subject or a group of subjects who are healthy or do not have a disease leading to immunodepression. It may also be an immunodepressed subject or group of subjects, for example following immunosuppressant treatment. Finally, it may be the same individual who underwent transplantation, for example before or just after the latter.

The reference level may be determined by a plurality of methods. For example, the control may be a predetermined value, which may assume various forms. It may be a unique threshold value, such as a median or a mean. The “reference level” may be a unique value, also applicable to each patient individually. Alternatively, the reference level may vary as a function of the specific subpopulations of patients. Thus, for example, older men might have a different reference level than younger men for the TTV load, and women might have a different reference level than men for this viral load. Moreover, the “reference level” may be established on the basis of comparative groups, such as groups who do not have a high level of TTV nucleic acid (or protein) and groups having high levels of TTV nucleic acid (or protein). Another example of comparative groups would be the groups having a disease, a condition or particular symptoms and the disease-free groups. The predetermined value may be defined, for example, when a test population is divided equally (or unequally) into groups, such as a low-risk group, a medium-risk group and a high-risk group.

The reference level may also be determined by comparing the level of TTV nucleic acid (or protein) in populations of patients who have undergone transplants or of patients with diseases leading to immunodepression. This may be accomplished, for example, by a histogram analysis, in which a whole cohort of subjects is presented graphically, with a first axis representing the level of said TTV nucleic acid (or protein), and a second axis representing the number of patients in the group of patients expressing TTV nucleic acid (or protein) at a given level. Two separate groups of subjects or more may be determined by identifying subpopulations of the cohort who have identical or similar levels of TTV nucleic acid (or protein). The reference level may then be determined on the basis of a level that best distinguishes these separate groups. A reference level may also represent the levels of two or more of the TTV nucleic acids (or proteins) present. Two markers or more may be represented, for example, by a ratio of values for the levels of each marker.

Moreover, a population apparently in good health will have a normal range “different” than that of a population known to have a state associated with a high concentration of said TTV nucleic acid (or protein). Consequently, the predetermined value selected may take into account the category into which an individual falls. Appropriate ranges and categories may be selected simply by means of routine experiments by a person skilled in the art. “High” or “raised” means high relative to a selected control. Generally, the control will be based on normal individuals apparently in good health in an appropriate age group.

In a preferred embodiment, the reference concentration corresponds to the concentration of TTV nucleic acid (or protein) or of the combination of TTV nucleic acids (or proteins) in the general population.

It will also be understood that the controls in the method described here may be, besides predetermined values, biological samples measured in parallel with the test samples. According to this embodiment, the reference level will be that of the TTV nucleic acid or acids (or protein or proteins) in a sample obtained from a subject in good health.

Preferably, the reference concentration of TTV nucleic acid (or protein) will be the concentration of this TTV nucleic acid (or this TTV protein) in a subject in good health or in a population of subjects in good health. According to another preferred embodiment, the reference concentration of the TTV nucleic acid (or protein) will be the concentration of this TTV nucleic acid (or this TTV protein) in an immunodepressed subject or in a population of immunodepressed subjects (for example, following an immunosuppressant treatment). According to another preferred embodiment, the reference concentration of the TTV nucleic acid (or protein) will be the concentration of this TTV nucleic acid (or this TTV protein) in the same individual who underwent a transplant at a specific time point, for example before or just after the latter.

Preferably, in the methods as described above, in all their embodiments, T cells are CD3+ T cells, CD4+ T cells, CD8+ T cells or a population of CD3+ and/or CD4+ and/or CD8+ T cells, preferably of CD3+ T cells.

### Method for Monitoring the Activity of the T Cells

The methods described here allow quick and easy evaluation of the proliferative capacity of the T cells in a subject.

Multiple factors contribute to the severe immunodepression status in recipients of HSCT, in particular of allo-HSCT. The conditioning alters in particular the recipient's lymphoid tissues. The presence of GvHD as well as its treatment cause new immunologic complications. Finally, the slight contingent of grafted T cells, compared to the size of T cell compartment in an immunocompetent person, as well as the extremely limited number of immune precursors of the donor present in the graft also contribute to the slowness of restoration of immunity in the recipient. All these factors make the recipient susceptible to posttransplant complications, such as microbial infections or GvHD.

Thanks to the methods described here, it is easily possible to evaluate the activity of the T cells in situations where the immune system has been compromised, for example such as after HSCT, especially allo-HSCT.

Another aspect of the present disclosure therefore relates to a method for monitoring the activity of the T cells in a patient who has received HSCT, especially allo-HSCT. This method comprises steps of:
a) measuring the proliferative capacity of the T cells at a first time point by the methods described above;
b) comparing the proliferative capacity of the T cells measured in a) with a reference proliferative capacity of the T cells; and
c) determining the change in the activity of the patient's T cells in view of the comparison in step b).

A “reference proliferative capacity of the T cells”, as it is understood here, is a proliferative capacity of the T cells estimated from a reference TTV load as described above. Of course, the comparison in step b) may be done simply by comparing the TTV load in the patient's sample determined in step a) with a reference TTV load.

For example, by comparing the proliferative capacity of the T cells in the patient at this first time point with the reference proliferative capacity of the T cells of an immunodepressed individual, it is possible to estimate, at this first time point, the restoration of the patient's immunocompetence after HSCT, especially allo-HSCT. Here, “immunocompetence” means acquisition of functionality by the immune cells. Thus, an increased proliferative capacity of the T cells in the patient relative to that of an immunodepressed individual indicates that restoration of the patient's immunocompetence is under way. As noted above, said increased proliferative capacity of the T cells in the patient relative to the immunodepressed subject corresponds to a lower TTV load.

Alternatively, the reference proliferative capacity of the T cells may be that of a healthy individual. In this case, a decreased proliferative capacity of the T cells in the patient relative to the reference proliferative capacity of the T cells indicates that immunocompetence has not been completely restored in the patient. It will be understood immediately that a decrease in the proliferative capacity of the T cells in the patient relative to the healthy subject corresponds to an increase in the TTV load.

The term “increased”, as used here in certain embodiments, signifies a larger amount, for example an amount slightly above the original amount, or for example an amount greatly in excess relative to the original amount, and in particular all the amounts in the range. As a variant, “increase” may refer to an amount or an activity that is at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, 90%, or 100% more than the amount or activity for which the increased amount or activity is compared. The terms “enhanced”, “greater than”, “larger” and “increased” are used interchangeably here.

The term “decreased”, as used here in certain embodiments, signifies a smaller amount, for example an amount slightly less than the original amount, or for example a very insufficient amount relative to the original amount, and in particular all the amounts in the range. As a variant, “decrease” may refer to an amount or an activity that is at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, 90%, or 100% less than the amount or activity for which the reduced amount or activity is compared. The terms “decreased”, “smaller than”, “lower” and “reduced” are used interchangeably here.

It is also possible to use, as reference proliferative capacity of the T cells, the proliferative capacity of the T cells in the same patient at a second time point. The changes in the activity of these T cells in this patient after transplant may thus be monitored easily, without using expensive and complicated testing equipment, by a simple measurement of the TTV load in the patient at the first and second time points.

According to this particular embodiment, the present method for monitoring the activity of the T cells in a patient who has received HSCT, especially allo-HSCT, comprises steps of:
a) measuring the proliferative capacity of the T cells at a first time point by the methods described above;
b) measuring the proliferative capacity of the T cells at a second time point by the methods described above, this second time point being later than the first time point in step a);
c) comparing the proliferative capacities of the T cells measured in a) and b); and
d) determining the change in the activity of the patient's T cells in view of the comparison in step c).

According to another particular embodiment, the method for monitoring the activity of the T cells in a subject who has received a transplant, preferably HSCT, and in particular an allo-HSCT, therefore comprises the steps of:
a) determining the TTV viral load from a biological sample of the subject taken at a first time point;
b) determining the TTV viral load from a biological sample of the subject taken at a second time point, this second time point being later than the first time point in step a);
c) comparing the viral loads of TTV measured in a) and b); and
d) determining the change in the subject's TTV load in view of the comparison in step c).

As explained above, since the TTV viral load is inversely correlated with the proliferative capacity of the T cells, the change in the TTV load makes it possible to determine whether the proliferative capacity of the T cells is increased or decreased, thus supplying an indication of the activity of the T cells and in particular regarding the restoration or otherwise of the subject's immunocompetence.

This method is thus particularly useful for monitoring the activity of the T cells in the patient over time. In a preferred embodiment, the first time point in step a) is located at the time of the transplant. In another preferred embodiment, the proliferative capacity of the T cells is measured in step b) using a sample taken at least 30 days, 60 days, 90 days, 100 days, 120 days, 150 days, 180 days, 210 days, 240 days, 270 days, 300 days, 330 days, 360 days, 720 days or 1080 days after HSCT. Alternatively, this sample is taken at 30 days, 60 days, 90 days, 100 days, 120 days, 150 days, 180 days, 210 days, 240 days, 270 days, 300 days, 330 days, 360 days, 720 days or 1080 days after HSCT.

It is clear that if the cells have an increased proliferative capacity at the second time point relative to the first, their activity is itself increased at this time point. For example, if the first time point is the time of the transplant, an increased proliferative capacity of the T cells at this second time point signifies that there are more T cells that are active and that the patient is then more able in particular to resist threats. These changes in the proliferative capacity of the T cells will translate into a change in the TTV load in the opposite direction: a decrease of the latter over time thus reflects an increase in the proliferative capacity of the T cells, i.e. restoration of the patient's immunocompetence. The present method therefore makes it possible to estimate the restoration of the recipient's immune system. In other words, the changes in the proliferative capacity of T cells make it possible to evaluate the reappearance of immunocompetence in the patient after transplant.

By monitoring the changes in the activity of T cells over time, it is possible in particular to monitor the restoration of immunocompetence after transplant in a patient who has undergone conditioning. According to this particular embodiment, the method for monitoring the activity of the T cells described above is employed in a patient who has undergone conditioning before receiving HSCT, especially allo-HSCT. In a preferred embodiment, the conditioning is myeloablative. In another preferred embodiment, the conditioning is attenuated.

### Methods of Evaluating the Risk of Microbial Infection

HSCT, in particular allo-HSCT, may lead to early or late complications, which vary considerably from patient to patient.

In particular, a patient who has received HSCT, especially allo-HSCT, remains sensitive to microbial infections, whether they are bacterial, viral, parasitic, or fungal, until their immune system is restored. By evaluating the proliferative capacity of the T cells at a given moment after HSCT, especially allo-HSCT, it is possible to determine the susceptibility to microbial infections of a patient who has undergone transplantation.

In this particular aspect, the present description relates to a method for determining the susceptibility to microbial infections in a patient who has received HSCT, especially allo-HSCT. This method comprises steps of:
a) measuring the proliferative capacity of the T cells from a sample from the patient by the methods described above at a first time point;
b) comparing the proliferative capacity of the T cells with a reference proliferative capacity of the T cells; and
c) determining the susceptibility to microbial infections in the patient from the comparison in step b).

By monitoring the changes in the activity of T cells over time, it is possible in particular to monitor the restoration of immunocompetence after transplant in a patient who has undergone conditioning. According to this particular embodiment, the method for monitoring the activity of the T cells described above is employed in a patient who has undergone conditioning before receiving HSCT, especially allo-HSCT. In a preferred embodiment, the conditioning is myeloablative. In another preferred embodiment, the conditioning is attenuated.

The methods described here may further comprise one or more steps of specific diagnostics of the presence of one or more of the infectious agents, for example such as bacteria, viruses, parasites or yeasts and filamentous fungi mentioned in more detail hereunder. Detection of these agents is routine clinical practice, in particular in connection with HSCT, and the corresponding techniques are familiar to a person skilled in the art. Therefore it is not necessary to detail them here.

These microbial infections are in particular viral, bacterial, parasitic, or fungal infections. The viral infections are in particular infections by viruses of the family Herpesviridae, for example such as the viruses HSV, VZV, HHV-6, Epstein-Barr virus (EBV) or human cytomegalovirus (HCMV). These viral infections may also be caused by adenoviruses, respiratory syncytial virus (RSV), influenza virus (also called influenzavirus or Myxovirus influenzae) or BK virus. The bacteria responsible for bacterial infections may be, among others, staphylococci such as Staphylococcus aureus or coagulase-negative staphylococci, encapsulated bacteria such as Streptococcus pneumoniae, Neisseria meningitidis or Haemophilus influenzae, Legionella sp. or also strictly aerobic nonfermentative Gram-negative bacilli such as the genera Pseudomonas, Acinetobacter, Stenotrophomonas, Burkholderia, Alkaligenes etc. Infections with atypical mycobacteria may also be observed. Parasitic infections of high morbidity and mortality, in particular with Pneumocystis carinii, may occur, and so may toxoplasmoses (caused by Toxoplasma gondii). Finally, yeasts such as Candida or Cryptococcus, but also filamentous fungi such as Aspergillus are responsible for invasive fungal infections that are among the major causes of infectious mortality after HSCT.

The reference proliferative capacity of the T cells corresponds to the proliferative capacity of the T cells determined from a reference TTV load as described above. The comparison in step b) may be done simply by comparing the TTV load in the patient's sample determined in step a) with a reference TTV load.

This reference TTV load may for example be that of a healthy individual who is not immunodepressed. The reference proliferative capacity of the T cells is then that of this healthy individual who is not immunodepressed.

In this particular embodiment, the description relates to a method for determining the susceptibility to microbial infections in a patient who has received HSCT, especially allo-HSCT. This method comprises steps of:
a) measuring the proliferative capacity of the T cells from a sample from the patient by the methods described above at a first time point;
b) comparing the proliferative capacity of the T cells with that of a healthy subject; and
c) determining the susceptibility to microbial infections in the patient from the comparison in step b).

By monitoring the changes in the activity of T cells over time, it is possible in particular to monitor the restoration of immunocompetence after transplant in a patient who has undergone conditioning. According to this particular embodiment, the method for monitoring the activity of the T cells described above is employed in a patient who has undergone conditioning before receiving HSCT, especially allo-HSCT. In a preferred embodiment, the conditioning is myeloablative. In another preferred embodiment, the conditioning is attenuated.

In this case, a reduced proliferative capacity of the T cells in the patient relative to the healthy subject indicates that the patient's immune system is not fully functional. A proliferative capacity of the T cells of the patient less than that of a healthy subject corresponds to a viral load measured in the patient's sample greater than the TTV load in the healthy subject. In other words, the subject then has a deficiency of his immune system, which leaves him open to attack by pathogens. Thus, the patient is at risk of being infected by microbial organisms. However, the patient is less susceptible to microbial infections when his T cells have substantially the same proliferative capacity as those of a subject in good health.

The proliferative capacity of the T cells in the same patient at a second time point may also be used as the reference proliferative capacity of the T cells. A person skilled in the art is thus able to monitor the evolution of the risks of microbial infections over time after transplant. Thus, the anti-infectious treatments may be adapted as a function of the patient's real susceptibility to the microbial infections, which limits the risks of appearance of resistance, while improving the patient's quality of life.

In this particular embodiment, the method of determining the susceptibility to microbial infections in a patient who has received HSCT, especially allo-HSCT, comprises steps of:
a) measuring the proliferative capacity of the T cells at a first time point by the methods described above;
b) measuring the proliferative capacity of the T cells at a second time point by the methods described above, this second time point being later than the first time point in step a);
c) comparing the proliferative capacities of the T cells measured in a) and b); and
d) determining the susceptibility to microbial infections in the patient in view of the comparison in step c).

By monitoring the changes in the activity of T cells over time, it is possible in particular to monitor the restoration of immunocompetence after transplant in a patient who has undergone conditioning. According to this particular embodiment, the method for monitoring the activity of the T cells described above is employed in a patient who has undergone conditioning before receiving HSCT, especially allo-HSCT. In a preferred embodiment, the conditioning is myeloablative. In another preferred embodiment, the conditioning is attenuated.

It is thus possible to follow the evolution over time of the susceptibility to microbial infections in the patient. An increased proliferative capacity of the T cells in step b) relative to step a) reflects an increase in their activity and therefore a decrease in the patient's susceptibility to the microbial infections between the two time points. The present method makes it possible in particular to verify that the more time passes after transplant, the less the patient is susceptible to microbial infections, i.e. his immune system becomes more and more functional.

The patient's susceptibility to microbial infections may therefore be determined using the methods described here, making it possible to adapt a treatment that is specific to the patient's needs. The preliminary determination of the patient's immunodepressed state with the method of the invention thus leads to a safer treatment than the treatments designed on the basis of the methods of the prior art.

Preferably, in the methods as described above, in all their embodiments, the proliferative capacity of the T cells corresponds to the proliferative capacity of the CD3+ T cells, CD4+ T cells, CD8+ T cells or of a population of CD3+ and/or CD4+and/or CD8+ T cells, preferably to the proliferative capacity of the CD3+ T cells.

Thus, the present invention also relates to a method of designing a treatment for microbial infections in a patient who has undergone HSC, in particular an allo-HSCT, said method comprising:
a) determining the patient's susceptibility to the microbial infections by the methods described above;
b) deciding on a treatment in accordance with the result from step a).

By monitoring the changes in the activity of T cells over time, it is possible in particular to monitor the restoration of immunocompetence after transplant in a patient who has undergone conditioning. According to this particular embodiment, the method for monitoring the activity of the T cells described above is employed in a patient who has undergone conditioning before receiving HSCT, especially allo-HSCT. In a preferred embodiment, the conditioning is myeloablative. In another preferred embodiment, the conditioning is attenuated.

The treatment may be decided preventively, i.e. it may be prescribed in view of the patient's immunodeficiency state, to prevent the occurrence of an infection. In this context, it may be decided to prescribe the therapeutic or prophylactic treatments commonly used after HSCT, especially allo-HSCT, which are described hereunder. The present methods thus make it possible to decide on and administer a prophylactic or therapeutic treatment of a viral, bacterial, parasitic, or fungal infection if a risk of such an infection is identified.

The present description therefore also relates to a method of treating an infection in a patient who has received HSCT, especially allo-HSCT, said method comprising steps of:
a) determining the patient's susceptibility to the microbial infections by the methods described above; and
b) administering a suitable treatment to said subject.

The present invention thus proposes a treatment intended to be used in the treatment of an infection in a subject who has received HSCT, especially allo-HSCT, said use comprising the steps of:
a) determining the patient's susceptibility to the microbial infections by the methods described above; and
b) administering a suitable treatment to said subject.

In other words, the invention relates to the use of a treatment in the preparation of a medicinal product for treating an infection in a subject who has received HSCT, especially allo-HSCT, said use comprising steps of:
a) determining the patient's susceptibility to the microbial infections by the methods described above; and
b) administering a suitable treatment to said subject.

By monitoring the changes in the activity of T cells over time, it is possible in particular to monitor the restoration of immunocompetence after transplant in a patient who has undergone conditioning. According to this particular embodiment, the method for monitoring the activity of the T cells described above is employed in a patient who has undergone conditioning before receiving HSCT, especially allo-HSCT. In a preferred embodiment, the conditioning is myeloablative. In another preferred embodiment, the conditioning is attenuated.

As explained above, the infections encountered after HSCT, especially allo-HSCT, are viral, bacterial, parasitic, or fungal infections. The treatments of these infections are well known and have been used in clinical practice for many years (see for example Tomblyn et al., Biol Blood Marrow Transplant. 15(10): 1143-1238, 2009). Thus, the viral infections may be prevented by antivirals such as aciclovir, ganciclovir, cidofovir, entecavir, fludarabine, lamivudine, tenofovir, ribavirin or valaciclovir, or specific monoclonal antibodies such as palivizumab (against RSV). The antibiotics usually allow bacterial infections to be treated. Broad-spectrum antibiotics are used in particular, such as beta-lactam antibiotics, glycopeptides, fosfomycin, macrolides, tetracyclines, aminoglycosides, chloramphenicol, quinolones, rifampicin and sulfamides. Regarding the parasitic infections, they are usually treated by administering antiparasitics such as cotrimoxazole, pyrimethamine and sulfadiazine.

Finally, the antifungals that may be administered are well known and comprise in particular fluconazole and the echinocandins.

Methods of evaluating the risk of graft-versus-host disease (GvHD)

The methods described here also offer the advantage of being able to estimate the risk of GvHD developing in a patient who has undergone HSCT, in particular allo-HSCT.

The present description therefore also relates to a method for determining the susceptibility to GvHD in a patient who has received HSCT, especially allo-HSCT, this method comprising steps of:
a) measuring the proliferative capacity of the T cells from a sample from the patient at a first time point by the methods described above; and
b) determining the susceptibility to GvHD in the patient from the measurement in step a).

By monitoring the changes in the activity of T cells over time, it is possible in particular to monitor the restoration of immunocompetence after transplant in a patient who has undergone conditioning. According to this particular embodiment, the method for monitoring the activity of the T cells described above is employed in a patient who has undergone conditioning before receiving HSCT, especially allo-HSCT.

In a preferred embodiment, the conditioning is myeloablative. In another preferred embodiment, the conditioning is attenuated.

“Graft-versus-host disease” or “GvHD”, as it is understood here, is an inflammatory immune reaction directed against the recipient and involving immunocompetent cells present in the graft. There are two clinical forms of GvHD: acute GvHD, which usually occurs in about the first 100 days following transplantation, and chronic GvHD, which generally occurs beyond this limit. Acute GvHD refers to the appearance of an allogenic inflammatory response in three organs exclusively: the skin, the liver and the gastrointestinal tract. In contrast, chronic GvHD may affect at least one of the following eight organs: skin, mouth, eyes, gastrointestinal tract, liver, lungs, muscles, joints, fascia and the genitalia. Whether for the acute form or the chronic form, GvHD is usually diagnosed by a clinical examination, which may comprise a histologic analysis of a biopsy of the organ in question (Schoemans et al. Bone Marrow Transplant 53: 1401-1415, 2018).

In this respect, it should be noted that the present method is particularly useful, for example because it predicts the possibility of discontinuing the immunosuppressant treatment of the patients and therefore ensuring their survival. The present method offers the possibility of easily differentiating an active GvHD that requires continuation of immunosuppressant treatment from a GvHD that is no longer at all active and for which the treatment could be stopped (cf. Magro et al., Bull Cancer. 104S: S145—S168, 2017).

More particularly, acute GvHD occurs in the first month (30 days) following transplantation, whereas chronic GvHD appears between 100 and 400 days after transplantation. Both are characterized by activation of the donor's T cells present in the graft. The interaction between the host's antigens and the donor's T cells leads to allogenic activation of the T cells, their proliferation and their differentiation into effector cells that attack the host's epithelial cells.

The recipient of HSCT, especially of allo-HSCT, is liable to develop a GvHD if the T cells in the patient's sample are capable of proliferating. In particular, proliferation of the patient's T cells at a time when the immune system is not yet restored is a strong indication that the patient is liable to be affected by GvHD. This can easily be evaluated by measuring the TTV load, using the methods described above. The indication given by this test may be supplemented if necessary by a clinical examination of the patient, in particular by histologic analysis of one or more biopsies from one or more of the patient's organs.

Preferably, the subject's sample is taken less than 400 days after transplantation. More preferably, the sample is taken less than 100 days after transplantation; alternatively, the sample is taken between 100 and 400 days after transplantation. In a particular embodiment, the GvHD is an acute GvHD; in another particular embodiment, the GvHD is a chronic GvHD.

In a particular embodiment, it may be useful to compare the measurement from step b) with a reference having a known proliferative capacity of the T cells, i.e. a reference proliferative capacity of the T cells. The reference proliferative capacity of the T cells corresponds to the proliferative capacity of the T cells estimated from a reference TTV load as described above. The comparison in step b) may be done simply by comparing the TTV load in the patient's sample determined in step a) with a reference TTV load.

This reference TTV load may for example be that of a healthy individual who is not immunodepressed. The reference proliferative capacity of the T cells is then that of this healthy individual who is not immunodepressed.

Alternatively, the reference TTV load may be that of an immunodepressed individual. In this particular embodiment, the description relates to a method for determining the susceptibility to GvHD in a patient who has received HSCT, especially allo-HSCT. This method comprises steps of:
a) measuring the proliferative capacity of the T cells from a sample from the patient by the methods described above at a first time point;
b) comparing the proliferative capacity of the T cells with that of an immunodepressed subject; and
c) determining the susceptibility to GvHD in the patient from the comparison in step b).

By monitoring the changes in the activity of T cells over time, it is possible in particular to monitor the restoration of immunocompetence after transplant in a patient who has undergone conditioning. According to this particular embodiment, the method for monitoring the activity of the T cells described above is employed in a patient who has undergone conditioning before receiving HSCT, especially allo-HSCT. In a preferred embodiment, the conditioning is myeloablative. In another preferred embodiment, the conditioning is attenuated.

In this case, an increased proliferative capacity of the T cells in the patient relative to the immunodepressed subject indicates that the patient's immune system is becoming functional again. A proliferative capacity of the patient's T cells greater than that of an immunodepressed subject corresponds to a viral load measured in the patient's sample lower than the TTV load in the immunodepressed subject. In other words, the subject then has active T cells, which may potentially attack the cells of the graft and trigger a GvHD. However, the patient is not likely to develop a GvHD when his T cells have substantially the same proliferative capacity as those of an immunodepressed subject.

It is also possible to use, as the reference proliferative capacity of the T cells, the proliferative capacity of the T cells in the same patient at a second time point. A person skilled in the art can thus monitor the evolution of the risk of occurrence of the GvHD over time after transplantation. Thus, an anti-GvHD treatment may be adapted as a function of the patient's real susceptibility to GvHD, which limits the risks of appearance of resistance, while improving the patient's quality of life.

In this particular embodiment, the method of determining the susceptibility to GvHD in a patient who has received HSCT, especially allo-HSCT, comprises steps of:
a) measuring the proliferative capacity of the T cells at a first time point by the methods described above;
b) measuring the proliferative capacity of the T cells at a second time point by the methods described above, this second time point being later than the first time point in step a);
c) comparing the proliferative capacities of the T cells measured in a) and b); and
d) determining the susceptibility to GvHD in the patient in view of the comparison in step c).

By monitoring the changes in the activity of T cells over time, it is possible in particular to monitor the restoration of immunocompetence after transplant in a patient who has undergone conditioning. According to this particular embodiment, the method for monitoring the activity of the T cells described above is employed in a patient who has undergone conditioning before receiving HSCT, especially allo-HSCT. In a preferred embodiment, the conditioning is myeloablative. In another preferred embodiment, the conditioning is attenuated.

It is thus possible to follow over time the evolution of the susceptibility to GvHD in the patient. An increased proliferative capacity of the T cells in step b) relative to step a) reflects an increase in their activity and therefore an increase in the patient's susceptibility to development of GvHD between the two time points. This risk is greater if it occurs soon after transplantation, i.e. at a time when the only active T cells are those of the donor and when there is therefore a risk of them attacking the organs of the host.

The patient's susceptibility to GvHD may therefore be determined using the methods described here, which makes it possible to elaborate a treatment specific to the patient's needs.

Thus, the present invention also relates to a method of designing a treatment of GvHD for a subject who has received HSC, in particular HSCT, said method comprising:
a) determining the patient's susceptibility to GvHD by the methods described above;
b) deciding on a treatment in accordance with the result from step a).

By monitoring the changes in the activity of T cells over time, it is possible in particular to monitor the restoration of immunocompetence after transplant in a patient who has undergone conditioning. According to this particular embodiment, the method for monitoring the activity of the T cells described above is employed in a patient who has undergone conditioning before receiving HSCT, especially allo-HSCT. In a preferred embodiment, the conditioning is myeloablative. In another preferred embodiment, the conditioning is attenuated.

The present description also relates to a method of treatment of GvHD in a patient who has received HSCT, especially allo-HSCT, said method comprising steps of:
a) determining the patient's susceptibility to GvHD by the methods described above; and
b) administering a suitable treatment to said subject.

The present invention thus proposes a treatment intended to be used in the treatment of GvHD in a subject who has received HSCT, especially allo-HSCT, said use comprising the steps of:
a) determining the patient's susceptibility to GvHD by the methods described above; and
b) administering a suitable treatment to said subject.

In other words, the invention relates to the use of a treatment in the preparation of a medicinal product for treating GvHD in a subject who has received HSCT, especially allo-HSCT, said use comprising steps of:
a) determining the patient's susceptibility to GvHD by the methods described above; and
b) administering a suitable treatment to said subject.

By monitoring the changes in the activity of T cells over time, it is possible in particular to monitor the restoration of immunocompetence after transplant in a patient who has undergone conditioning. According to this particular embodiment, the method for monitoring the activity of the T cells described above is employed in a patient who has undergone conditioning before receiving HSCT, especially allo-HSCT. In a preferred embodiment, the conditioning is myeloablative. In another preferred embodiment, the conditioning is attenuated.

The treatments of GvHD are well known and have been the subject of recommendations on the part of clinicians (see for example, Magro et al., BullCancer. 104S: S145—S168, 2017; Penack et al., Lancet Haematol. 7(2): e157-e167; 2020). The treatments of GvHD may be used prophylactically and most often consist of immunosuppressant treatments such as ciclosporin or tacrolimus.

When the treatments of GvHD are used therapeutically, they vary as a function of the severity of the complication. However, these treatments most generally comprise immunosuppressants, corticoids, in particular prednisolone and methylprednisolone. Antilymphocyte serum (ALS) is also used if the corticoids fail. Finally, other second-line drugs may be used, such as mycophenolate mofetil (Cellcept®), monoclonal antibodies (anti TNFα or IL2 antireceptors).

The description also relates to the use of measurement of the variation of the TTV load in a subject for determining the proliferative capacity of the T cells of said subject.

The variation in the load may be determined by comparing the TTV load measured in a sample taken at a first time point and in a sample taken at a second time point, the second time point being later than the first.

As explained above, if the TTV load increases over time, this reflects, through a decrease in the proliferative capacity of the T cells, a decrease in the activity of said T cells. Conversely, a decrease in the TTV load over time reflects, through the increase in the proliferative capacity of the T cells, an increase in the activity of said T cells.

The present description also relates to the embodiments hereunder:

Embodiment 1: Method for determining the proliferative capacity of the T cells in a subject, the method comprising steps of:
a) measuring the TTV load from a biological sample of said subject; and
b) determining the proliferative capacity of the T cells in view of the viral load measured in a).

Embodiment 2: Method according to embodiment 1, characterized in that the TTV load is measured by amplification, sequencing or hybridization of a TTV sequence, preferably by amplification, more preferably by real-time PCR.

Embodiment 3: Method according to embodiment 1 or 2, characterized in that the biological sample is a sample of whole blood, plasma or serum.

Embodiment 4: Method according to any one of the embodiments 1 to 3, characterized in that the determination in step b) comprises comparing the TTV load measured in a) with a reference TTV load.

Embodiment 5: Method according to any one of the embodiments 1 to 4, characterized in that the patient has received a transplant.

Embodiment 6: Method according to embodiment 5, characterized in that the patient has received a hematopoietic stem cell transplantation (HSCT), preferably an allo-HSCT.

Embodiment 7: Method according to embodiment 5 or 6, characterized in that the patient has undergone a conditioning, preferably myeloablative or attenuated, before transplantation.

Embodiment 8: Method for monitoring the activity of the T cells in a patient who has received an allo-HSCT, the method comprising steps of:
a) measuring the proliferative capacity of the T cells in the patient at a first time point according to any one of the embodiments 1 to 6;
b) comparing the proliferative capacity of the T cells measured in a) with a reference proliferative capacity of the T cells; and
c) determining the change in the activity of the patient's T cells in view of the comparison in step b).

Embodiment 9: Method of determining the susceptibility to microbial infections in a patient who has received an allo-HSCT, the method comprising steps of:
a) measuring the proliferative capacity of the T cells in the patient at a first time point according to any one of the embodiments 1 to 6;
b) comparing the proliferative capacity of the T cells measured in a) with a reference proliferative capacity of the T cells; and
c) determining the patient's susceptibility to microbial infections in view of the comparison in step b).

Embodiment 10: Method according to embodiment 9, characterized in that the microbial infection is a viral, bacterial, protozoon or fungal infection.

Embodiment 11: Method of determining the susceptibility to graft-versus-host disease (GvHD) in a patient who has received an allo-HSCT, the method comprising steps of:
a) measuring the proliferative capacity of the T cells in the patient at a first time point according to any one of the embodiments 1 to 6;
b) comparing the proliferative capacity of the T cells measured in a) with a reference proliferative capacity of the T cells; and
c) determining the patient's susceptibility to GvHD in view of the comparison in step b).

Embodiment 12: Method according to any one of the embodiments 8 to 11, characterized in that the reference proliferative capacity of the T cells is the proliferative capacity of the T cells of a healthy individual or the proliferative capacity of the T cells of an immunodepressed individual.

Embodiment 13: Method according to any one of the embodiments 8 to 11, characterized in that the reference proliferative capacity of the T cells is the proliferative capacity of the T cells measured in the patient at a second time point.

The invention will be described more precisely by means of the examples given hereunder.

FIG. 1: Representation of the structure of the genome of a TTV isolate. Organization of the genome of a prototype TTV (TTV-1a isolate). The arrows represent the major ORFs (with a length greater than 50 amino acids). The GC-rich region and a region N22 (from which the TTV was isolated originally) are indicated. The untranslated region UTR corresponds to the region from the 3′ end of ORF4 to the 5′ end of ORF2. From Biagini, Curr Top Microbiol lmmunol. 331: 21-33, 2009.

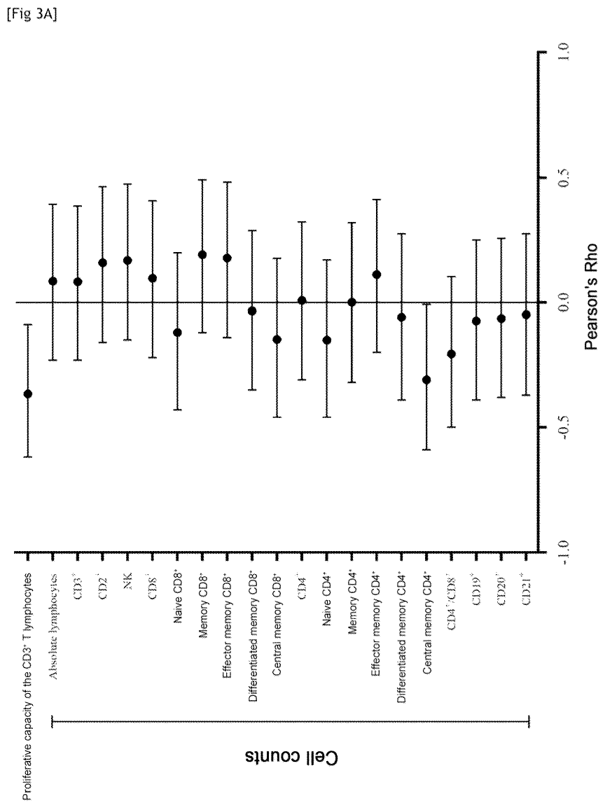

FIG. 2: TTV viral load from plasma samples from recipients of allo-HSCT and from healthy volunteers. The TTV viral load of 41 recipients of allo-HSCT (black) and of 54 healthy volunteers (white) was quantified. After extraction of the DNA, the TTV viral load was quantified using the TTV R-GENE® kit (available only for research and not for diagnostics, Ref#69-030, bioMérieux. Marcy-I'Etoile, France). The lowest viral load detected was 0.46 Log copy/mL (log cp/mL). The log copy/mL are used for describing the expression of the viral load of the TTV between the two populations. The variance was compared using an F-test (## p<0.01). The mean TTV viral load (black line) was compared using an unpaired t-test with the Welch correction (*** p<0.001).

Abbreviations: DNA. Deoxyribonucleic acid; Allo. allogenic; HSCT. Hematopoietic stem cell transplantation; TTV. torque teno virus.

The proliferative capacity of the CD3+ T cells from 41 recipients of allo-HSCT (black) and from 20 healthy volunteers (white) was quantified after stimulation for 3 days with a mitogen (PHA) and was measured by flow cytometry using the Click-It® EdU AF488 kit. The variance of the two populations was compared using an F-test (## p<0.01). Comparison of the mean values (black line) was performed using an unpaired t-test with Welch correction (***. p<0.001).

Abbreviations: Allo, allogenic; HSCT, Hematopoietic stem cell transplantation; PHA, Phytohaemagglutinin.

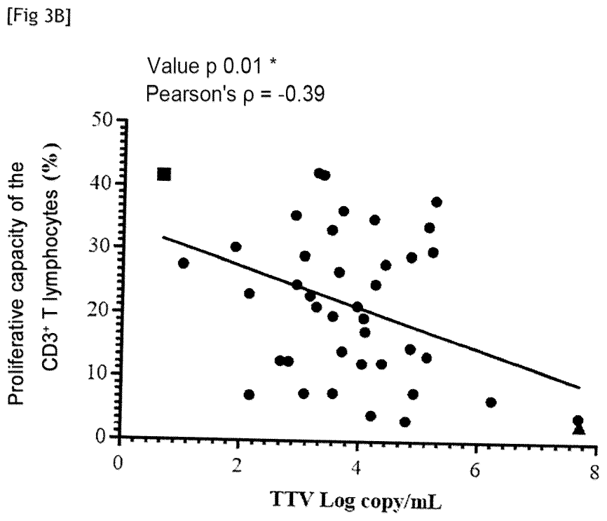

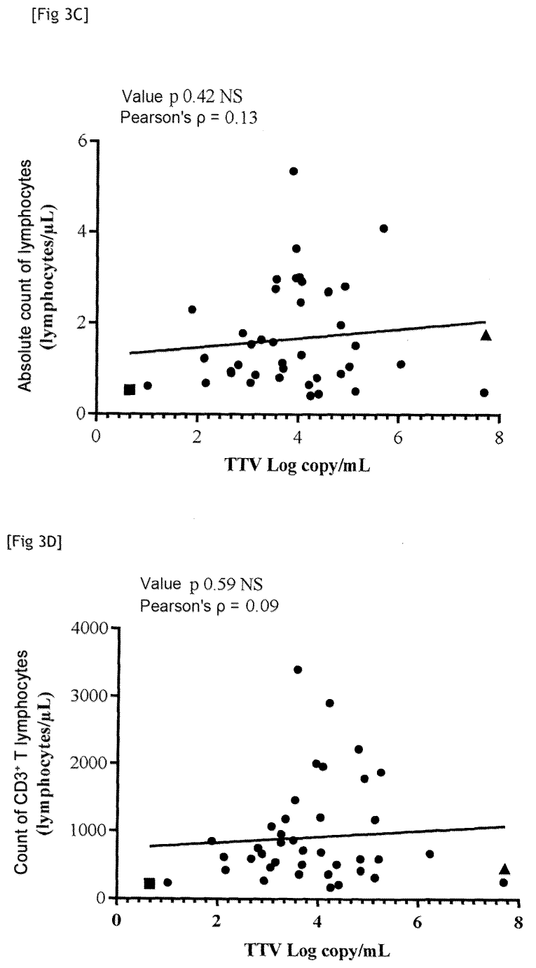

FIGS. 3A-D: Correlation between the TTV viral load and T cell count as well as the proliferative capacity of the CD3⁺ T cells. Overall correlation of the TTV viral load of plasma from 41 recipients of allo-HSCT relative to the count of several T cell subtypes and relative to the proliferative capacity of the CD3⁺ T cells (A). Pearson's rho and the 95% confidence interval (CI95) for all the parameters evaluated are represented by a black dot and a black line, respectively.

Detailed correlation of the TTV viral load expressed in Log copies/mL of plasma from 41 recipients of allo-HSCT as a function of: (B) the proliferative capacity of the CD3⁺ T cells, (C) the absolute lymphocyte count and (D) the count of CD3⁺ T cells. The patients are represented by dots. The extreme patients: “A” (square) and “B” (triangle), as well as linear regression (black line) are shown.

The number of lymphocytes was measured by flow cytometry in the immunology laboratory using a broad panel of T cell membrane markers. The proliferative capacity of the CD3⁺ T cells was determined after 3 days of stimulation with a mitogen (PHA) and was measured by flow cytometry using the Click-It® EdU AF488 kit. The correlation between the TTV viral load (x axis) and the number of lymphocytes or the proliferative capacity of the CD3⁺ T cells (y axis) was determined using the Pearson correlation coefficient (indicated on each diagram).

Abbreviations: Allo. Allogenic; HSCT. Hematopoietic stem cell transplantation; NK. Natural killer; PHA. Phytohaemagglutinin; TTV. Torque teno virus,

Overall correlation of the plasma TTV viral load expressed in Log cp/mL obtained from 41 plasmas of allo-HSCT recipients relative to the immunophenotyping of the T cells and to the proliferative capacity of the CD3⁺ T cells (A). Detailed correlation of the TTV viral load expressed in Log cp/mL of 41 plasmas of allo-HSCT recipients relative to the proliferative capacity of the CD3⁺ T cells (B), absolute number of lymphocytes (C) and the number of CD3+ T cells (D). The subtypes and the absolute number of lymphocytes were measured by flow cytometry in the immunology laboratory of the Edouard Herriot hospital (Hospices Civils de Lyon) using a broad panel of membrane markers of the T cells. The proliferative capacity of the CD3+was determined after stimulation for 3 days with a mitogen (PHA) and was measured by flow cytometry using the Click-It® EdU AF488 flow kit. The correlation of the TTV viral load (abscissa) and number of cells or the proliferative capacity of CD3+ T cells (ordinate) was determined using the Pearson correlation coefficient (indicated on each diagram). (A) The values −0.5 and 0.5 represented by black dotted lines correspond to the limits of the correlation confidence interval. Pearson's rho and the 95% confidence interval (Cl) for all the parameters are represented by dots and a black line, respectively. (B, C and D) The patients are represented by black dots, the extreme patients by a square and a triangle; the linear regression is represented by a black line.

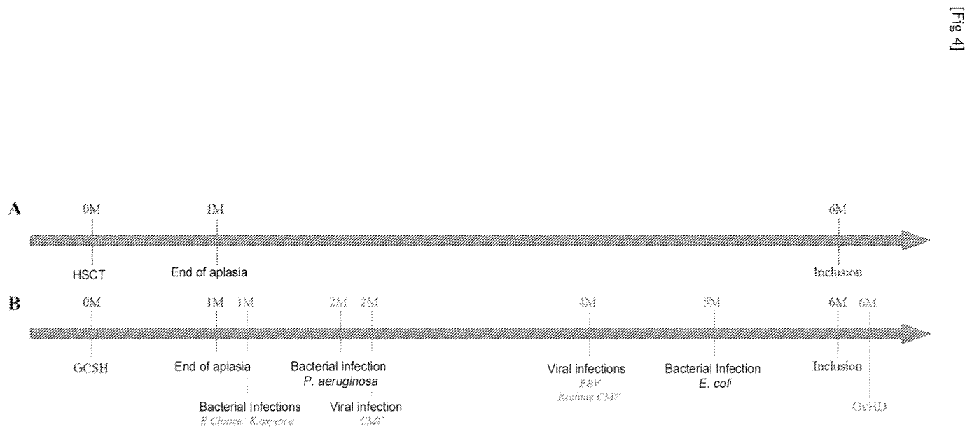

FIG. 4: Chronological descriptive monitoring of the patients with extreme values of TTV viral load. Chronological description of the main clinical stages (in black) and of the infectious episodes (in gray) between HSCT and inclusion for patient “A” and patient “B”. Patient “A” had the lowest TTV viral load and conversely patient “B” had the highest TTV viral load.

Abbreviations: CMV. Cytomegalovirus; EBV. Epstein-Barr virus; HSCT. Hematopoietic stem cell transplantation; GvHD. Graft-versus-host disease; M. Months.

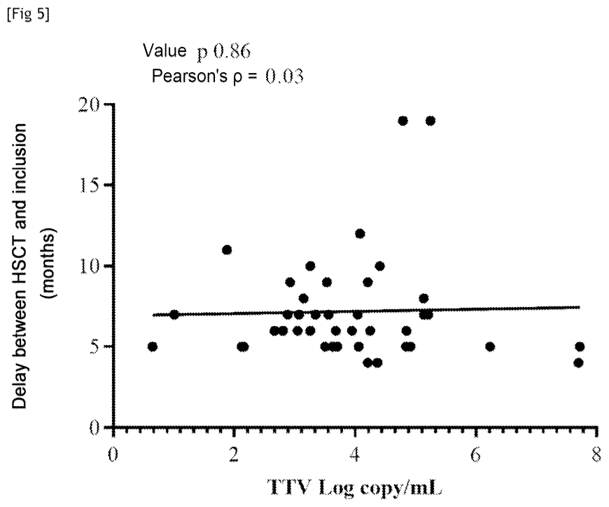

FIG. 5: Correlation between the TTV viral load and the times since HSCT. Detailed correlation between the plasma TTV viral load expressed in Log cp/mL of 41 plasmas of allo-HSCT recipients and the time lapse between HSCT and inclusion expressed in months. The correlation of the TTV viral load (abscissa) and the delays (ordinate) was determined using the Pearson correlation coefficient (indicated on each diagram). The patients are represented by black dots and the linear regression is represented by a black line.

Abbreviations: HSCT, hematopoietic stem cell transplantation; TTV, Torque Teno Virus.

### EXAMPLES

### Example 1

In our study, we evaluated and compared the correlation between the TTV viral load with the number of immune cells and the function of the immune cells in the period of post-transplantation restoration of immunity of the allogenic-HSCT (allo-HSCT) recipients.

### Materials and Methods

### The Study Population

Samples of heparinized whole blood and plasma samples treated with EDTA obtained from patients who were receiving an allo-HSCT were obtained from the prospective cohort Vaccheminf, described previously (13). The cohort was approved by a regional examining committee (Comité de protection des personnes Sud-Est V, Grenoble, France, number 69HCL17_0769) and is registered in ClinicalTrial.gov (NCT03659773). Consecutive adult patients who had undergone an allo-HSCT in the hematology department of CHU Lyon were included prospectively, once the patient's written consent had been obtained.

On admission, the data collected such as demographic characteristics (age, sex) and the clinical data (type of transplant, immunophenotyping, immunosuppressant treatment, GvHD status and GvHD treatment) were recorded using an electronic case report form (eCRF).

In parallel, 80 healthy individuals (HV) were recruited from the donors of the Lyon blood bank (Etablissement Francais du Sang, EFS). According to the standarized procedures of the EFS for blood donation and the provisions of articles R.1243-49 ff. of the public health code, a written non-objection to the use of the donated blood for research purposes was obtained from people in good health. The age and the sex of the blood donors were sent anonymously to the research laboratory. The regulatory authorizations for the handling and storage of these samples were obtained from the regional ethics committee (Comité de protection des personnes Sud-Est II, Bron, France) and the French ministry of research (Ministry of Higher Education, Research and Innovation, Paris, France).

50 μL of elution volume of viral DNA was extracted from 200 μl of the plasma samples using an easyMag extractor (bioMérieux, France) following the manufacturer's instructions. The presence of TTV and the TTV load were then determined using the TTV R-GENE® kit (bioMérieux, Marcy-l′Etoile, France) as described previously (14,15).

### T Cell Proliferation Test

Peripheral blood mononuclear cells (PBMC) were isolated from the heparinized fresh blood samples (heparinized whole blood) by Ficoll density gradient centrifugation (U-04; Eurobio, Les Ulis, France). Next, 10⁵ cells/well were incubated for 24 hours in a supplemented culture medium in a 96-well cell culture plate at 37° C. at 5% CO₂ (RPMI 1640; Eurobio). The PBMCs were then stimulated twice with a mitogen, phytohaemagglutinin (PHA) at 4 μg/mL (R30852801; Remel, Oxoid, Thermo Fisher Scientific, USA) and were incubated for 72 hours. The culture supernatants of the PBMCs were recovered for performing an IFNγ secretion assay (IGRA, for “IFNγ-release assay”) using Simple Plex cartridges on the ELLA nanofluidic system (ProteinSimple, San Jose, CA, USA) in accordance with the manufacturer's instructions. The proliferation of T cells was analyzed in the pellets with the Click-iT™ Plus EdU Alexa Fluor™ 488 flow cytometry assay kit (C10420; Life Technologies, Carlsbad, CA, USA), which measures the incorporation of 5-ethynyl-2′-deoxyuridine (EdU), in accordance with the published protocol (16). Briefly, the percentage of EdU⁺ proliferating cells (among the CD3⁺ cells) was found by flow cytometry analyses carried out on a BD LSR FortessaTM flow cytometer (BD Biosciences, San Jose, CA, USA). At least 2.5×10³ CD3+cells were measured for each experiment. The data were analyzed using the BD FACSDiva software (version 8.0.3, BD Biosciences). Immunophenotyping of T cells post-transplantation

The leukocytes and the CD4⁺ and CD8⁺ T cells were counted in the immunology laboratory of the Edouard Herriot hospital (Hospices Civils de Lyon). In addition, a broad panel of membrane markers of T cells was measured by flow cytometry on whole blood. The following were counted in this way: the naive CD4⁺ and CD8+ T cells (CD45+CCR7+), the central memory CD4⁺ and CD8+ T cells (CD45RA⁻ CCR7⁺), the effector memory CD4⁺ and CD8⁺ T cells (CD45⁻ CCR7⁺) and the differentiated memory CD4⁺ and CD8+ T cells (CD45RA⁺ CCR7⁻), as described previously (14). The results were expressed in cells/A.

### Statistical Analysis

The immunophenotyping data, the TTV viral load and the proliferative capacity of T cells are expressed as the mean value (range). The TTV load converted to Log format was used for the analysis (Log copy/mL). The differences between the healthy and allo-HSCT recipients were calculated using an unpaired parametric t test with the Welch correction. The correlations were evaluated using a parametric Pearson rho correlation coefficient. Regression analyses were carried out to evaluate the association between the dependent variable (TTV viral load) and the independent variables (percentage of proliferating cells, absolute number of lymphocytes and number of CD3+ T cells). The analysis of variance was carried out using F tests. The differences in plasma TTV load with respect to various clinical characteristics were determined using the Mann-Whitney test. A value of p <0.05 was considered significant. The statistical analyses were performed using the GraphPad Prism® software (version 5; GraphPad software, La Jolla, CA, USA) and R (version 3.5.1).

### Results

### Characteristics of the Cohort

Healthy volunteers (n=80) and allo-HSCT recipients (n=41) were enrolled between May 2018 and April 2020. The individuals in good health and the recipients of allo-HSCT did not differ significantly in terms of age (median [IR]: 56 [40-64] vs 46 [31-53] years, respectively) and sex (sex ratio: 1.6 vs 1.4, respectively). The median duration [IR] after transplantation at enrollment was 6 [5-8] months for the recipients of allo-HSCT. At inclusion, 78% of the recipients of allo-HSCT received immunosuppressant drugs (corticoids, calcineurin inhibitors, others . . . ), and 17% had a chronic graft-versus-host disease [Table 1].

TTV viral load in the plasma samples from healthy volunteers and allo-HSCT

The TTV viral load was studied in plasma samples obtained from 80 healthy recipients and 41 allo-HSCT recipients. The TTV viral load was detected by real-time PCR in 68% of the healthy samples (54/80). Regarding the allo-HSCT recipients, all the patients included in the study had a detectable value of TTV viral load. The mean (range) TTV viral load was significantly higher in the allo-HSCT recipients relative to the healthy subjects (3.9 (0.7-7.7) vs 2.1 (0.5-4.3) Log copy/ml respectively, p<0.0001) [FIG. 2].

Correlation between the TTV viral load, the numbers of T cells and proliferative capacity of the latter.

When the allo-HSCT recipients included 6 months after transplantation were taken into consideration, most of the population of lymphocyte subtypes was within ranges of normal values (NV). However, the quantities of T cells CD4⁺, naive CD4⁺, central memory CD4⁺, effector memory CD4⁺ in terminal differentiation, naive CD8⁺ and central memory CD8⁺, as well as the CD4⁺/CD8⁺ ratio, were below the normal values (see [Table 1]).

TABLE 1
Baseline characteristics of the allo-HSCT recipients All the laboratory
data were recorded at enrollment of the recipients.

| Composition | AL n = 41 | |
|---|---|---|
| Age, median [IR] | 56 | [40-64] |
| Men, n (%) | 24 | (59) |
| Hematologic and transplantation characteristics, | | |
| n (%) | | |
| * Underlying hematologic diseases | | |
| Myeloid tumors and acute leukemias | 37 | (90) |
| Acute myeloid leukemia and associated | 22 | (54) |
| tumors | | |
| Lymphoma/lymphoblastic leukemia with | 5 | (12) |
| B precursors | | |
| Lymphoma/lymphoblastic leukemia with | 1 | (2) |
| T precursors | | |
| Myelodysplastic/myeloproliferative | 1 | (2) |
| tumors | | |
| Myelodysplastic syndromes | 7 | (17) |
| Tumor with plasmacytoid dendritic blast | 1 | (2) |
| cells | | |
| Lymphoid, histiocytic, and mature dendritic | 4 | (10) |
| tumors | | |
| Hodgkin disease | 1 | (2) |
| Tumors with mature B cells, mature T | 3 | (7) |
| cells or NK cells | | |
| CR before transplant, n (%) | 39 | (95) |
| Types of donors | | |
| Geno-identical | 17 | (41) |
| Haplo-identical | 6 | (15) |
| Pheno-identical | 18 | (44) |
| Total compatibility | 15 | (37) |
| HLA incompatibility | 3 | (7) |
| Sources of stem cells | | |
| Peripheral blood cells | 28 | (68) |
| Bone marrow | 13 | (32) |
| Conditioning | | |
| MAC | 17 | (41) |
| RIC | 24 | (59) |
| TBI | 11 | (27) |
| Posttransplant complications, n (%) | | |
| Acute GvHD | 30 | (73) |
| Grade I/II | 21/9 | |
| Chronic GvHD | 7 | (17) |
| Limited/extended | 5/2 | |
| Time since transplant in months (median [IR]) | 6 | [5-8] |
| Immunophenotyping, median [IR] | | |
| Total lymphocytes (NV, 1000-2800/μL) | 1659 | (410-5350) |
| CD3⁺ T cells (NV, 521-1772/μL) | 915 | (175-3406) |
| CD3⁺ CD4⁺ T cells (NV, 336-1126/μL) | 273 | (38-876) |
| Naive CD4⁺ (CD45⁺CCR7⁺) (NV, 121-456/μL) | 45 | (0-445) |
| Central memory CD4⁺ (CD45RA⁻CCR7⁺) (NV, | 60 | (1-168) |
| 92-341/μL) | | |
| Effector memory CD4+ (CD45RA⁻CCR7⁻) (NV, | 163 | (4-522) |
| 59-321/μL) | | |
| Effector memory CD4⁺ in terminal differentiation | 19 | (0-147) |
| (CD45RA⁺CCR7⁻) (NV, 11-102/μL) | | |
| CD3⁺ CD8⁺ T cells (NV, 125-780/μL) | 602 | (60-2779) |
| Naive CD8⁺ (CD45⁺CCR7⁺) (NV, 86-257 μL) | 40 | (0-241) |
| Central memory CD8⁺ (CD45RA⁻CCR7⁺) (NV, | 17 | (0-127) |
| 19-93/μL) | | |
| Effector memory CD8⁺ (CD45RA⁻CCR7⁻) (NV, | 286 | (0-1517) |
| 15-162/μL) | | |
| Effector memory CD8⁺ in terminal differentiation | 257 | (0-1474) |
| (CD45RA⁺CCR7⁻) (NV, 39-212/μL) | | |
| CD4⁺/CD8⁺ ratio (NV, 0.9-6) | 0.84 | (0.13-8.88) |
| B cells CD20⁺ (NV, 64-593/uL) | 287 | (14-1299) |
| Posttransplant immunomodulator treatment at | | |
| enrollment, n (%) | | |
| #IS treatment | 32 | (78) |
| Corticosteroids | 5 | (12) |
| IVIG administration | 23 | (56) |
| Time since last IVIG administration, in months | 4 | [2-5] |
| (median [IR]) | | |
| DLI | 7 | (17) |
| ECP | 2 | (5) |

Abbreviations: Allo, allogenic; DLI, donor lymphocyte infusion; GvHD, graft-versus-host disease; HLA, human leukocyte antigen; HSCT, hematopoietic stem cell transplantation; IR, interquartile range; TBI, total body irradiation; IS, immunosuppressant; IVIG, intravenous polyclonal immunoglobulins; MAC, myeloablative conditioning; NK, Natural Killer; ECP extracorporeal photochemotherapy; CR, complete remission; RIC, reduced intensity conditioning; NV, normal values.
* According to the 2016 revision of the World Health Organization classification of myeloid and lymphoid neoplasms.
#Immunosuppressant treatments included: antithymocyte globulins, ciclosporin, tacrolimus, methotrexate, mycophenolate mofetil, cyclophosphamide, corticosteroids ≥ 1 mg/kg > 21 days. The subtypes and the total number of lymphocytes were measured by flow cytometry in the immunology laboratory of the Edouard Herriot hospital (Hospices Civils de Lyon) using a broad panel of membrane markers of T cells.
The normal values indicated are supplied by the laboratory of the Edouard Herriot hospital (Hospices Civils de Lyon).

Relative to the healthy subjects, besides a significantly lower proliferative capacity among the CD3⁺ cells in the allo-HSCT recipients (40.5% vs 21.3% respectively, p<0.0001), a greater and more significant heterogeneous distribution may also be noted (of [2.9% to 42.3%] and [29.7% to 55.3%] for allo-HSCT and healthy subjects respectively; F test p=0.0040), underlying the interindividual variability in the restoration of immunity of the allo-HSCT recipients (not shown graphically).

Using the Pearson correlation test (rho [C195]), the highest correlation was observed between the TTV viral load and the proliferative capacity of T cells [FIG. 3A] and [FIG. 3B]. It should be noted that no significant correlation was observed with the total number of lymphocytes or a particular subset of cells (for example CD3±) (Pearson's rho ρ=−0.39 [CI95% −0.62 to −0.09]), vs (ρ=0.13 [−0.19 to 0.42]) and (ρ=0.09 [−0.23 to 0.38]) respectively) [FIG. 3C] and [FIG. 3D].

### Clinical Characteristics of the Patients with Extreme Values of TTV Load

On analyzing, at the individual level, the correlation between the TTV load and the proliferative capacity of T cells in response to PHA stimulation, we noted that the patient (A, represented by a square) with the lowest viral load (0.65 Log copy/ml) had a high percentage of proliferating cells (41.6%) and conversely the patient (B, represented by a triangle) with the highest viral load (7.72 Log copy/ml) had a low percentage of proliferating cells (2.9%) [FIG. 3B]. These two patients (a man and a woman) were in the same age group (50<age<60) and were in complete remission before their transplantation. Patient (A) had received a transplant of stem cells from a geno-identical donor whereas patient (B) had received a transplant of peripheral blood cells from a pheno-identical donor. Patient (A) had a simple posttransplant evolution without particular clinical events between transplantation and enrollment, i.e. no infectious episode, no GvHD and no immunosuppressant treatment [FIG. 4]. Conversely, patient (B) had received onerous immunosuppressant treatment and suffered acute GvHD and multiple severe bacterial/viral infections [FIG. 4].

### Discussion

First we compared the prevalence of TTV and the viral loads in the plasma of two distinct populations, 80 healthy immunocompetent subjects and 41 immunodepressed allo-HSCT transplants of the monocentric prospective cohort VaccHemInf (13).

According to a recent study (18), a prevalence of 68% of TTV was observed in the samples from healthy subjects. In comparison, TTV was found in 100% of the samples from immunodepressed patients. We have also confirmed that the plasma viral load of TTV was significantly higher in the recipients of allo-HSCT relative to the HV (10,19). Six months after allo-HSC transplant, some patients are therefore unable to regulate the TTV viral load despite a sufficient number of T cells. No correlation was found between the post-transplantation delay [5-8 months] and the plasma TTV viral load. One of the main results from this study is that the plasma viral load of TTV was significantly higher for the allo-HSCT recipient (at 6 months after transplantation) relative to the healthy subjects, thus confirming the observations of Tyagi et al., 2013 (post-transplantation delay not stated) or Masouridi et al., 2016 (2-3 months after transplant) (10,18). This result might be explained by the fact that T cells, the main cells of the immune response against viral infection (19,20), are one of the principal sites of replication of TTV (21,22). In the allo-HSCT recipients at 6 months after transplant, multiplication of the T cells is under way, since the immune system is being restored, and this supplies a large number of cells where the virus can replicate. It has also been described that the TTV load is greatest about 3 to 6 months after transplantation before returning to a so-called normal value (23,24). It might therefore be hypothesized that the TTV would utilize the growth of T cells, still naive and nonfunctional, to replicate before being finally regulated by the immune system and by the functional cells, thus suggesting an important link between TTV viral load and immune function. This study included evaluation of the correlation between plasma TTV load and a quantitative marker, the immune cell count, already evaluated in several studies but with contradictory results (17,23-25), and a qualitative marker of the restoration of immunity, measurement of proliferation of T cells after a nonspecific stimulation. No effect of the period between allo-HSCT transplantation and enrollment was observed [FIG. 5]. Despite the heterogeneity of the distribution of proliferation value of the heterogeneous T cells, which emphasizes the interindividual variability of an immunodepressed population, we observed a greater correlation between TTV viral load and the proliferation of T cells than with the immune cell count [FIG. 3A]. More precisely, it is interestingly an inverse correlation, suggesting that the greater the number of functional immune cells, the lower the TTV load. This result is consistent with those described in 2013 by De Vlaminck et al. (11) in transplantation of solid organs, where the decrease in the level of immunosuppression was correlated with the decrease in TTV viral load. This also corresponds to the many descriptions of the kinetics of TTV viral load after transplantation (23,26), in which a phase of decrease connected with the immunosuppressant treatments is observed initially, followed by a growth phase associated with expansion of the immune cells competent for replication of the TTV, then a phase of stabilization and finally a decrease in viral load to a baseline level, reflecting a functional immune restoration. It could be thought that, 6 months after allo-HSCT transplant, there would be a sufficient number of immune cells in expansion to allow significant replication of the TTV, but they would not be sufficiently functional to regulate the TTV viral load. Immunodepressed patients would therefore be unable to regulate the TTV viral load despite a sufficient number of T cells. This is also the case with other viruses classically described in the recipients of allo-HSCT such as the cytomegalovirus (CMV) or the Epstein-Barr virus (EBV) (24,27,28). In our cohort, 24% and 37% of our patients, respectively, were infected with these two viruses. This is also consistent with the observations made for our patients with extreme values of TTV viral load.

The TTV viral load has also been linked to the number of CD8⁺/CD57⁺ T cells, a subtype of lymphocyte described as a potential marker of immunosenescence and increasing in the course of certain pathologies such as acquired immunodeficiency states, transplants or persistent viral infections. All these results suggest that there is therefore a potentially important link between the TTVs and the function of the immune system, in particular with respect to its capacity to regulate the viral load.

In our study, no effect was observed of various clinical characteristics (for example state, underlying disease, immunosuppressant treatment or GvHD) on the TTV load ([Table 2]).

TABLE 2
Comparison of the plasma TTV load with the clinical characteristics
of 41 recipients of allo-HSCT. All the data were recorded
at the time of enrollment of the recipient.

| | | TTV Log cp/mL | p- |
|---|---|---|---|
| | n (%) | median [IR] | value |
| Sex | | | |
| Men | 24 (59) | 3.7 [2.9-4.8] | 0.38 |
| Women | 17 (41) | 4.1 [3.5-4.4] | |
| Hematologic diseases | | | |
| Myeloid tumors and acute leukemias | 37 (90) | 3.9 [3.1-4.4] | 0.28 |
| Lymphoid, histiocytic, and mature | 4 (10) | 5.0 [3.8-5.8] | |
| dendritic tumors | | | |
| Types of donor | | | |
| Geno-identical | 17 (41) | 3.7 [3.2-4.3] | 0.52 |
| Haplo-identical | 6 (15) | 4.4 [3.3-5.1] | |
| Pheno-identical | 18 (44) | 4.1 [3.2-4.7] | |
| Sources of stem cells | | | |
| Peripheral blood cells | 28 (68) | 4.0 [3.1-4.8] | 0.43 |
| Bone marrow | 13 (32) | 3.7 [3.2-4.1] | |
| Conditioning | | | |
| MAC | 17 (41) | 3.6 [2.9-4.2] | 0.22 |
| RIC | 24 (59) | 4.1 [3.1-4.9] | |
| TBI | | | |
| Yes | 11 (27) | 3.7 [2.9-4.5] | 0.46 |
| No | 30 (73) | 4.1 [3.1-4.6] | |
| GVHD | | | |
| Yes | 31 (76) | 3.7 [3.0-4.8] | 0.4 |
| No | 10 (24) | 4.1 [3.8-4.3] | |
| GVHD status | | | |
| Acute | 30 (73) | 3.9[2.9-4.8] | 0.65 |
| Chronic | 7 (17) | 3.6 [3.0-4.2] | |
| Immunosuppressant treatment | | | |
| Yes | 32 (78) | 3.9 [3.0-4.4] | 0.54 |
| No | 9 (22) | 4.0 [3.2-4.9] | |
| IVIG administration | | | |
| Yes | 23 (56) | 3.9 [3.0-4.5] | 0.59 |
| No | 18 (44) | 4.1 [3.2-4.7] | |

Abbreviations: GvHD, graft-versus-host disease; IR, interquartile range; IVIG, intravenous polyclonal immunoglobulins; MAC, myeloablative conditioning; RIC, reduced intensity conditioning; TBI, total body irradiation; TTV, Torque Teno Virus.
* According to the 2016 revision of the World Health Organization classification of myeloid and lymphoid neoplasms.
# Immunosuppressant treatments included: antithymocyte globulins, ciclosporin, tacrolimus, methotrexate, mycophenolate mofetil, cyclophosphamide, corticosteroids ≥ 1 mg/kg > 21 days.
The median plasma TTV load [IR] was compared with the clinical data using the Mann-Whitney test (***, p < 0.001).

To summarize, this study establishes the existence of a correlation between TTV viral load and T cell function, and shows that this correlation is independent of the number of cells.

### Example 2

Comparison between the number of T cells, the proliferative capacity of said T cells and the plasma TTV viral load between several patients receiving allogenic-HSCT (allo-HSCT) from example 1.

Quantification of the TTV viral load, the proliferation test and counting of CD3+ T cells were carried out according to the same protocols as example 1. The results are presented in Table 3 below.

TABLE 3
Comparison of the plasma TTV viral load, the number of CD3+
T cells and the proliferative capacity of said lymphocytes.

| | TTV viral load | Number of CD3+ T cells | Proliferative |
|---|---|---|---|
| | (log copies/mL) | (cells/μL) | capacity (%) |
| Patient 1 | 3.26 | 832 | 21.14 |
| Patient 2 | 3.94 | 2011 | 21.32 |
| Patient 3 | 0.65 | 222 | 41.60 |
| Patient 4 | 7.70 | 262 | 4.08 |

Comparing the TTV viral load and the number of CD3+ T cells, it can be seen that for a similar viral load in the patients, the number of CD3+ T cells may be significantly different (Patient 1 vs Patient 2) and conversely, for a significantly different viral load (Patient 3 vs Patient 4), the number of CD3+ T cells may be similar.

Consequently, this confirms that the TTV viral load is not correlated with the number of CD3+ T cells.

However, comparing the number of CD3+ T cells and the proliferative capacity, it can be seen that in patients having a similar proliferative capacity of the CD3+ T cells, the number of said lymphocytes may be significantly different (Patient 1 vs Patient 2). Conversely, when the number of CD3+ T cells is similar, the proliferative capacity may be significantly different (Patient 3 vs Patient 4).

Consequently, this confirms that the number of CD3+ T cells is not exclusively correlated with their proliferative capacity, and that a high number of CD3+ T cells is no indication of the post-stimulation proliferative capacity of said lymphocytes and therefore of the functionality of the immune system, in contrast to the TTV viral load.

In fact, looking at the TTV viral load, it can be seen that it is inversely correlated with the proliferative capacity of the CD3+ T cells (Patient 3 vs Patient 4).

### REFERENCES

1. Nishizawa T, Okamoto H, Konishi K, Yoshizawa H, Miyakawa Y, Mayumi M. A Novel DNA Virus (TTV) Associated with Elevated Transaminase Levels in Posttransfusion Hepatitis of Unknown Etiology. Biochem Biophys Res Commun. dec 1997;241(1):92-7.
2. Focosi D, Antonelli G, Pistello M, Maggi F. Torquetenovirus: the human virome from bench to bedside. Clin Microbiol Infect. juill 2016;22(7):589-93.
3. Spandole S, Cimponeriu D, Berca LM, Mihăescu G. Human anelloviruses: an update of molecular, epidemiological and clinical aspects. Arch Virol. avr 2015;160(4):893-908.
4. de Villiers E-M, Borkosky S S, Kimmel R, Gunst K, Fei J-W. The diversity of torque teno viruses: in vitro replication leads to the formation of additional replication-competent subviral molecules. J Virol. juill 2011;85(14):7284-95.
5. Kosulin K, Kernbichler S, Pichler H, Lawitschka A, Geyeregger R, Witt V, et al. Post-transplant Replication of Torque Teno Virus in Granulocytes. Front Microbiol. 2018;9:2956.
6. Maggi F, Bendinelli M. Immunobiology of the Torque Teno Viruses and Other Anelloviruses. In: de Villiers E-M, Hausen H zur, éditeurs. TT Viruses [Internet]. Berlin, Heidelberg: Springer Berlin Heidelberg; 2009 [cité 20 août 2019]. p. 65-90. Disponible sur: http://link.springercom/10.1007/978-3-540-70972-5_5
7. Hino S, Miyata H. Torque teno virus (TTV): current status. Rev Med Virol. fevr 2007;17(1):45-57.
8. Mitchell A B, Glanville A R. Kinetics of TTV-DNA Plasma Load: A Global Measure of Immune Suppression? Transplantation. avr 2019;103(4):660-1.
9. Focosi D, Maggi F, Albani M, Macera L, Ricci V, Gragnani S, et al. Torquetenovirus viremia kinetics after autologous stem cell transplantation are predictable and may serve as a surrogate marker of functional immune reconstitution. J Clin Virol. fevr 2010;47(2):189-92.
10. Masouridi-Levrat S, Pradier A, Simonetta F, Kaiser L, Chalandon Y, Roosnek E. Torque teno virus in patients undergoing allogeneic hematopoietic stem cell transplantation for hematological malignancies. Bone Marrow Transplant. mars 2016;51(3):440-2.
11. De Vlaminck I, Khush K K, Strehl C, Kohli B, Luikart H, Neff N F, et al. Temporal Response of the Human Virome to Immunosuppression and Antiviral Therapy. Cell. nov 2013;155(5):1178-87.
12. Hoshina T, Ohga S, Fujiyoshi J, Nanishi E, Takimoto T, Kanno S, et al. Memory B-Cell Pools Predict the Immune Response to Pneumococcal Conjugate Vaccine in Immunocompromised Children. J Infect Dis. 1 mars 2016;213(5):848-55.
13. Conrad A, Boccard M, Valour F, Alcazer V, Tovar Sanchez A-T, Chidiac C, et al. VaccHemInf project: protocol for a prospective cohort study of efficacy, safety and characterisation of immune functional response to vaccinations in haematopoietic stem cell transplant recipients. BMJ Open. févr 2019;9(2):e026093.
14. Kulifaj D, Durgueil-Lariviere B, Meynier F, Munteanu E, Pichon N, Dube M, et al. Development of a standardized real time PCR for Torque teno viruses (TTV) viral load detection and quantification: A new tool for immune monitoring. J Clin Virol. aoOt 2018;105:118-27.
15. Kulifaj D, Essig M, Meynier F, Pichon N, Munteanu E, Moulinas R, et al. Torque teno virus (TTV) in immunosuppressed host: Performances studies of TTV R-Gene® kit and donors and recipients kidney samples genotyping. J Clin Virol. sept 2016;82:S103-4.
16. Poujol F, Monneret G, Friggeri A, Rimmelé T, Malcus C, Poitevin-Later F, et al. Flow cytometric evaluation of lymphocyte transformation test based on 5-ethynyl-2′deoxyuridine incorporation as a clinical alternative to tritiated thymidine uptake measurement. J Immunol Methods. déc 2014;415:71-9.
17. Focosi D, Spezia PG, Macera L, Salvadori S, Navarro D, Lanza M, et al. Assessment of prevalence and load of torquetenovirus viraemia in a large cohort of healthy blood donors. Clin Microbiol Infect. janv 2020;S1198743X20300367.
18. Tyagi A, Pradier A, Baumer O, Uppugunduri C R S, Huezo-Diaz P, Posfay-Barbe K M, et al. Validation of SYBR Green based quantification assay for the detection of human Torque Teno virus titers from plasma. Virol J. 2013;10(1):191.
19. Rosendahl Huber S, van Beek J, de Jonge J, Luytjes W, van Baarle D. T cell responses to viral infections - opportunities for Peptide vaccination. Front Immunol. 2014;5:171.
20. Sant AJ, McMichael A. Revealing the role of CD4+ T cells in viral immunity. J Exp Med. 30 juill 2012;209(8):1391-5.
21. Focosi D, Macera L, Boggi U, Nelli LC, Maggi F. Short-term kinetics of torque teno virus viraemia after induction immunosuppression confirm T cells as the main replication-competent cells. J Gen Virol. 1 janv 2015;96(Pt_1):115-7.
22. Maggi F, Fornai C, Zaccaro L, Morrica A, Vatteroni ML, Isola P, et al. TT virus (TTV) loads associated with different peripheral blood cell types and evidence for TTV replication in activated mononuclear cells. J Med Virol. juin 2001;64(2):190-4.
23. Albert E, Solano C, Giménez E, Focosi D, Pérez A, Macera L, et al. Kinetics of Alphatorquevirus plasma DNAemia at late times after allogeneic hematopoietic stem cell transplantation. Med Microbiol Immunol (Berl). avr 2019;208(2):253-8.
24. Wohlfarth P, Leiner M, Schoergenhofer C, Hopfinger G, Goerzer I, Puchhammer-Stoeckl E, et al. Torquetenovirus Dynamics and Immune Marker Properties in Patients Following Allogeneic Hematopoietic Stem Cell Transplantation: A Prospective Longitudinal Study. Biol Blood Marrow Transplant. janv 2018;24(1):194-9.
25. Schmitz J, Kobbe G, Kondakci M, Schuler E, Magorsch M, Adams O. The Value of Torque Teno Virus (TTV) as a Marker for the Degree of Immunosuppression in Adult Patients after Hematopoietic Stem Cell Transplantation (HSCT). Biol Blood Marrow Transplant. November 2019;S1083879119307426.
26. Gilles R, Herling M, Holtick U, Heger E, Awerkiew S, Fish I, et al. Dynamics of Torque Teno virus viremia could predict risk of complications after allogeneic hematopoietic stem cell transplantation. Med Microbiol Immunol (Berl). October 2017;206(5):355-62.
27. Ljungman P, Hakki M, Boeckh M. Cytomegalovirus in hematopoietic stem cell transplant recipients. Hematol Oncol Clin North Am. févr 2011;25(1):151-69.
29. Liu L, Zhang X, Feng S. Epstein-Barr Virus-Related Post-Transplantation Lymphoproliferative Disorders After Allogeneic Hematopoietic Stem Cell Transplantation. Biol Blood Marrow Transplant. juill 2018;24(7):1341-9.
29. Bosch M, Khan F M, Storek J. Immune reconstitution after hematopoietic cell transplantation: Curr Opin Hematol. juill 2012;19(4):324-35.

1. A method for determining a proliferative capacity of the T cells in a subject, comprising the steps of:
a) measuring a Torque Teno Virus (TTV) load from a biological sample of the subject; and
b) determining the proliferative capacity of the T cells in view of a viral load measured in a).

2. The method of claim 1, wherein the TTV load is measured by amplification, sequencing or hybridization of a TTV sequence.

3. The method of claim 1, wherein the biological sample is a sample of whole blood, plasma or serum.

4. The method claim 1, wherein the determination in step b) comprises comparing the TTV load measured in a) with a reference TTV load.

5. The method of claim 1, wherein the subject has received a transplant.

6. The method of claim 5, wherein the subject has received a hematopoietic stem cell transplantation (HSCT).

7. The method of claim 5, wherein the subject has undergone myeloablative or attenuated conditioning.

8. A method for monitoring activity of T cells in a subject who has received an allo-HSCT, comprising the steps of:
a) measuring a proliferative capacity of the T cells in the subject at a first time point by a method comprising the steps of
a1) measuring Torque Teno Virus (TTV) load from a biological sample of the subject; and
a2) determining the proliferative capacity of the T cells in view of the viral load measured in a1)
b) comparing the proliferative capacity of the T cells measured in a) with a reference proliferative capacity of the T cells; and
c) determining a change in the activity of the T cells of the subject in view of the comparison in step b).

9. A method for monitoring activity of T cells in a patient who has received a transplant, comprising the steps of:
a) determining a TTV viral load from a biological sample taken from the patient at a first time point;
b) determining the TTV viral load from a biological sample taken from the patient at a second time point, this second time point being later than the first time point in step a);
c) comparing the TTV viral loads measured in a) and b); and
d) determining the change in the patient's TTV load in view of the comparison in step c).

10. A method of determining susceptibility to microbial infections in a subject who has received an allo-HSCT, comprising the steps of:
a) measuring a proliferative capacity of T cells in the subject at a first time point by a method comprising the steps of
a1) measuring Torque Teno Virus (TTV) load from a biological sample of the subject; and
a2) determining the proliferative capacity of the T cells in view of the viral load measured in a1)
b) comparing the proliferative capacity of the T cells measured in a) with a reference proliferative capacity of the T cells; and
c) determining the susceptibility to microbial infections of the subject in view of the comparison in step b).

11. The method of claim 10, wherein the microbial infection is a viral, bacterial, protozoon or fungal infection.

12. A method of determining susceptibility to graft-versus-host disease (GvHD) in a subject who has received an allo-HSCT, comprising the steps of:
a) measuring a proliferative capacity of T cells in the subject at a first time point by a method comprising the steps of:
a1) measuring Torque Teno Virus (TTV) load from a biological sample of the subject; and
a2) determining the proliferative capacity of the T cells in view of the viral load measured in a1)
b) comparing the proliferative capacity of the T cells measured in a) with a reference proliferative capacity of the T cells; and
c) determining the subject's susceptibility to GvHD in view of the comparison in step b).

13. The method of claim 8, wherein the reference proliferative capacity of the T cells is the proliferative capacity of the T cells of a healthy individual or the proliferative capacity of the T cells of an immunodepressed individual.

14. The method of claim 8, wherein the reference proliferative capacity of the T cells is the proliferative capacity of the T cells measured in the subject at a second time point.

15. (canceled)

16. The method of claim 9, wherein the subject has received an allo-HSCT.

17. The method of claim 9, wherein the reference proliferative capacity of the T cells is the proliferative capacity of the T cells of a healthy individual or the proliferative capacity of the T cells of an immunodepressed individual.

18. The method of claim 10, wherein the reference proliferative capacity of the T cells is the proliferative capacity of the T cells of a healthy individual or the proliferative capacity of the T cells of an immunodepressed individual.

19. The method of claim 12, wherein the reference proliferative capacity of the T cells is the proliferative capacity of the T cells of a healthy individual or the proliferative capacity of the T cells of an immunodepressed individual.

20. The method of claim 9, wherein the reference proliferative capacity of the T cells is the proliferative capacity of the T cells measured in the subject at a second time point.

21. The method of claim 10, wherein the reference proliferative capacity of the T cells is the proliferative capacity of the T cells measured in the subject at a second time point.

22. The method of claim 12, wherein the reference proliferative capacity of the T cells is the proliferative capacity of the T cells measured in the subject at a second time point.


SEQUENCE LISTING (1 sequence)

SEQ ID NO: 1 - DNA, 3853 bp, Torque teno virus
attttgctacgtcactaaccacgtgacacccacaggccaaccgaatgctatgtcatccatttcctgggccgggtctacgtcctcatataagtaagtgcacttccgaatggctgagttttccacgcccgtccgcagcggtgaagccacggagggagatctccgcgtcccgagggcgggtgccgaaggtgagtttacacaccgaagtcaaggggcaattcgggctcgggactggccgggctatgggcaaggctctgaaaaaagcatgtttattggcaggcattacagaaagaaaagggcgctgtcactgtgtgctgtgcgaacaacaaagaaggcttgcaaactactaatagtaatgtggaccccacctcgcaatgatcaacactaccttaactggcaatggtactcaagtatacttagctcccacgctgctatgtgcgggtgtcccgacgctgtcgctcattttaatcatcttgcttctgtgcttcgtgccccgcaaaacccaccccctcccggtccccagcgaaacctgcccctccgacggctgccggctctcccggctgcgccagaggcgcccggagatagagcaccatggcctatggctggtggcgccgaaggagaagacggtggcgcaggtggagacgcagaccatggaggcgccgctggaggacccgaagacgcagacctgctagacgccgtggccgccgcagaaacgtaaggagacgccgcagaggagggaggtggaggaggagatataggagatggaaaagaaagggcaggcgcagaaaaaaagctaaaataataataagacaatggcaaccaaactacagaaggagatgtaacatagtaggctacatccctgtactaatatgtggcgaaaatactgtcagcagaaactatgccacacactcagacgataccaactacccaggaccctttggggggggtatgactacagacaaatttactttaagaattctgtatgacgagtacaaaaggtttatgaactactggacagcatctaacgaagacctagacctttgtagatatctaggagtaaacctgtactttttcagacacccagatgtagattttatcataaaaattaataccatgcctccttttctagacacagaactcacagcccctagcatacacccaggcatgctagccctagacaaaagagcaagatggatacctagcttaaaatctagaccgggaaaaaaacactatattaaaataagagtaggggcaccaagaatgttcactgataaatggtacccccaaacagatctttgtgacatggtgcttctaactgtctatgcaaccgcagcggatatgcaatatccgttcggctcaccactaactgactctgtggttgtgaacttccaggttctgcaatccatgtatgataaaacaattagcatattaccagacgaaaaatcacaaagagaaattctacttaacaagatagcaagttacattcccttttataataccacacaaactatagcccaattaaagccatttatagatgcaggcaatgtaacatcaggcgcaacagcaacaacatgggcatcatacataaacacaaccaaatttactacagcaaccacaacaacttatgcatatccaggcaccaacagacccccagtaactatgttaacctgtaatgactcctggtacagaggaacagtatataacacacaaattcaacagttaccaataaaagcagctaaattatacttagaggcaacaaaaaccttgctaggaaacaccttcacaaatgaggactacacactagaatatcatggaggactgtacagctcaatatggctatcccctggtagatcttactttgaaacaacaggagcatatacagacataaagtacaatccattcacagacagaggagaaggcaacatgttatggatagactggctaagcaaaaaaaacatgaactatgacaaagtacaaagtaaatgcttaatatcagacctacctctatgggcagcagcatatggatatgtagaattttgtgcaaaaagtacaggagaccaaaacatacacatgaatgccaggctactaataagaagtccctttacagacccacaactactagtacacacagaccccacaaaaggctttgttccttactctttaaactttggaaatggtaaaatgccaggaggtagtagtaatgtgcctattagaatgagagctaaatggtatccaacattatttcaccagcaagaagtactagaggccttagcacagtcaggcccctttgcataccactcagacattaaaaaagtatctctgggtatgaaataccgttttaagtggatctggggtggaaaccccgttcgccaacaggttgttagaaatccctgcaaagaaacccactcctcgggcaatagagtccctagaagcttacaaatcgttgacccgaaatacaactcaccggaactcacattccatacctgggacttcagacgtggcctctttggcccgaaagctattcagagaatgcaacaacaaccaacaactactgacattttttcagcaggccgcaagagacccaggagggacaccgaggtgtaccactccagccaagaaggggagcaaaaagaaagcttacttttccccccagtcaagctcctcagacgagtccccccgtgggaagactcgcagcaggaggaaagcgggtcgcaaagctcagaggaagagacgcagaccgtctcccagcagctcaagcagcagctgcagcaacagcgaatcctgggagtcaaactcagactcctgttcaaccaagtccaaaaaatccaacaaaatcaagatatcaaccctaccttgttaccaagggggggggatctagcatccttatttcaaatagcaccataaacatgtttggtgaccccaaaccttacaacccttccagtaatgactggaaagaggagtacgaggcctgtagaatatgggacagaccccccagaggcaacctaagagatacccctttctacccctgggcccccaaggaaaaccagtaccgtgtaaactttaaacttggattccaataaagctaggccgtgggactttcacttgtcggtgtctgcttataaaagtaactaagcactccgagcgaagcgaggagtgcgacccttgggggctcaacgccttcggagccgcgcgctacgccttcggctgcgcgcggcacctcagacccccgctcgtgctgacacgctcgcgcgtgtcagaccacttcgggctcgcgggggtcgggaaatttactaaacagactccgagttgccattggactcaggagctatgaatcagtaacgaaagtgagtggggccagacttcgccataaggcctttatcttcttgccatttgtcagtaacaggggtcgccatagacttcggcctccactttaccttgtaaaaactaccaaaatggccgttccagtgacgtcacagccgccattttaagtagctgacgtcaaggattgacgtaaaggttaaaggtcatcctcggcggaagctacacaaaatggtggacaacatcttccgggtcaaaggttgtgcgtacgtcacaagtcacgtggaggggacccgctgtaacccggaagtaggccccgtcacgtgacttaccacgtgtgtacacgtcaccgccgccattttgttttacaaaatggctgacttccttcctcttttttgaaaaaaggcgccaaaaaaccgtcggcgggggggccgcgcgctgcgcgcgcggcccccggggggaggcattgcctcccccccccgcgcgcatgcgcgcgggtccccccccctccggggggctccgccccccggcccccccc